(19) United States
(12) Patent Application Publication
(10) Pub. No.: US 2026/0347861 A1
(43) Pub. Date: Nov. 5, 2026

(54) FREQUENCY MODULATED CONTROL OF OPTOGENETICS AND CELLULAR FUNCTIONS

(71) Applicant: University of Southern California, Los Angeles, CA (US)

(72) Inventors: Yingxiao WANG, Pasadena, CA (US); Phuong HO, Los Angeles, CA (US)

(73) Assignee: University of Southern California, Los Angeles, CA (US)

(21) Appl. No.: 19/667,106

(22) Filed: May 4, 2026

Related U.S. Application Data
(60) Provisional application No. 63/798,762, filed on May 2, 2025.

(51) Int. Cl. C12N 15/63; C07K 14/435; C07K 14/705

(52) U.S. Cl. CPC C12N 15/635; C07K 14/43595; C07K 14/705; C07K 14/70503

(57) ABSTRACT

A system for frequency-modulated control of cellular functions in a mammalian cell includes an optogenetic circuit having one or both of a first light-responsive protein interaction module comprising a dimerization system responsive to high-frequency blue light (dense input) and a second light-responsive protein interaction module comprising a combined dissociation and dimerization system with differential reversion kinetics responsive to low-frequency blue light (sparse input). The optogenetic circuit decodes a frequency of light input and generates distinct intracellular outputs corresponding thereto, including at least one of frequency-dependent protein-protein interactions through operatively linked protein interaction domains, selective gene transcription via a synthetic gene construct having regulatory elements responsive to high-frequency or low-frequency stimulation, or frequency-dependent cell-cell communication through an operatively linked cell-surface signaling component.

### CROSS-REFERENCE TO RELATED APPLICATIONS

This application claims the benefit of U.S. provisional application Ser. No. 63/798,762 filed May 2, 2025, the disclosure of which is hereby incorporated in its entirety by reference herein.

### STATEMENT REGARDING FEDERALLY SPONSORED RESEARCH OR DEVELOPMENT

This invention was made with Government support under R01EB029122; R01 HL121365; R35 GM140929; and HD107206 awarded by the National Institutes of Health (NIH). The Government has certain rights in the invention.

### TECHNICAL FIELD

In at least one aspect, the present disclosure is related to synthetic biology and cellular engineering, and more particularly to optogenetic systems for frequency-modulated control of cellular functions. The disclosure pertains to methods and systems for using light-responsive genetic and protein interaction circuits to decode dynamic light input signals, such as variations in pulse frequency, in order to regulate intracellular processes, including protein complex formation, gene transcription, and cell-cell communication in mammalian cells.

### BACKGROUND

Biomolecular signaling networks allow cells to convert external input signals into the appropriate biological outputs^{1,2}. While it is common for a signal to be decoded based on its identity or amplitude, cells can also sense and respond to dynamic characteristics such as the pulse frequency^{3,4}. This mode of signaling, known as frequency modulation, is prevalent throughout cell biology. Pulsatile dynamics can be found in a wide range of biological processes, including immune cell activation, cell cycle progression, and intercellular communication⁵⁻¹⁰. The ability to decode pulsatile dynamics enables cells to fine-tune their responses to external stimuli, highlighting the importance of FM signaling in maintaining precise control over various biological outcomes.

FM signals enable the temporal control of signaling pathways, providing a powerful means of dynamically regulating genetic and cellular functions^{11,12}. When combined with a remote control system such as optogenetics, FM signaling offers a versatile platform for the regulation of complex cellular behaviors via a single modality. In mammalian cells, native systems such as the ERK signaling pathway facilitate differential interpretation of pulsed signals among various cellular subpopulations, giving rise to the formation of global tissue patterns^{13,14}. In contrast, many current synthetic systems lack the ability to selectively distinguish between multiple signaling states, providing only simple “off/on” switching responses to external stimuli¹⁵⁻¹⁸. Thus, the development of an optogenetic platform that can leverage FM decoding would enable more precise and complex signaling capabilities for applications in mammalian cells.

Accordingly, there is a need for an engineered optogenetic system capable of decoding frequency-modulated (FM) light inputs into distinct intracellular responses, thereby enabling frequency-specific control over cellular functions.

### SUMMARY

In at least one aspect, a system for frequency-modulated (FM) control of cellular functions in a mammalian cell is provided. This system includes an optogenetic circuit comprising one or both of a first light-responsive protein interaction module and a second light-responsive protein interaction module. The first module comprises a dimerization system that is responsive to high-frequency blue light stimulation, herein referred to as dense input. The second module comprises a combined dissociation and dimerization system with differential reversion kinetics, which is responsive to low-frequency blue light stimulation, referred to as sparse input. The optogenetic circuit is configured to decode the frequency of the light input and to generate distinct intracellular outputs corresponding to that frequency. The cellular outputs include at least one of: (i) frequency-dependent control of protein-protein interactions through protein interaction domains operatively linked to the optogenetic circuit; (ii) selective gene transcription via a synthetic gene construct comprising one or more regulatory elements that respond specifically to high-frequency or low-frequency stimulation; or (iii) frequency-modulated control over cell-cell communication through a cell-surface signaling component operatively linked to the optogenetic circuit. Advantageously, the protein interaction domains enabling frequency-dependent control of protein-protein interactions and the cell-surface signaling component enabling frequency-dependent control over cell-cell communication.

In another aspect, the system overcomes the limitations of existing synthetic circuits by providing the ability to discriminate between multiple dynamic input states—not merely their presence or absence—and translate these into functionally meaningful outputs such as protein-protein interactions, gene transcription, and intercellular communication. In particular, an FM-responsive optogenetic platform would provide a powerful and modular tool for applications in cell biology, regenerative medicine, immunotherapy, and synthetic biology by allowing multiplexed control of cellular behavior using a single input modality. The present disclosure addresses this need by providing a computationally designed and experimentally validated system that can interpret frequency variations in blue light illumination and generate specific, tunable biological outputs in mammalian cells.

In at least one aspect, a method for frequency-modulated control of cellular functions in a mammalian cell is provided. The method includes providing the mammalian cell with an optogenetic circuit comprising one or both of a first light-responsive protein interaction module and a second light-responsive protein interaction module. The first light-responsive protein interaction module comprises a dimerization system. The second light-responsive protein interaction module comprises a combined dissociation and dimerization system with differential reversion kinetics. The method further includes exposing the mammalian cell to blue light at a first frequency to selectively activate the first light-responsive protein interaction module and generate a first intracellular output. The method also includes exposing the mammalian cell to blue light at a second frequency that is different from the first frequency. The second frequency selectively activates the second light-responsive protein interaction module and generates a second intracellular output that is distinct from the first intracellular output.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The patent or application file contains at least one drawing executed in color. Copies of this patent or patent application publication with color drawing(s) will be provided by the Office upon request and payment of the necessary fee.

FIG. 1A. A system component block diagram showing the hierarchical arrangement of components of the system 10 for frequency-modulated control of cellular functions. The system 10 includes a light source 18 providing input to an optogenetic circuit 12 within a mammalian cell 38. The optogenetic circuit 12 includes a dense decoder 14 (first module) having a dimerization system 40, and a sparse decoder 16 (second module) having a combined dissociation and dimerization system 42. Outputs from the optogenetic circuit 12 are directed to protein interaction domains 20, a synthetic gene construct 22, and a cell-surface signaling component 24. The synthetic gene construct 22 includes a DNA-binding domain 26, a transcriptional activator 28, a dual promoter system 34, a fluorescent reporter construct 30, and a tumor-associated antigen 36. Downstream elements include synNotch receptors 44, receiver cells 46, and sender cells 48. A computationally optimized signaling motif 50 underlies the design of the optogenetic circuit 12 and is configured as an FM decoder 32.

FIG. 1B. A flowchart depicting a method for computationally optimizing an FM decoder motif. An initial population of model candidates with randomized parameters is generated (S100). Each candidate is evaluated for performance and simplicity (S110). Pareto selection identifies non-dominated candidates along the optimality front (S120). Mutation and crossover operations generate offspring candidates (S130). A determination is made whether the maximum number of generations has been reached (S140). If the maximum number of generations has not been reached, the process returns to evaluation (S110). If the maximum number of generations has been reached, the optimized FM decoder motif is output (S150).

FIGS. 2A, 2B, 2C, 2D, 2E, 2F, and 2G. Computational modeling and multi-objective optimization of an FM decoder motif. (A) An overview of the desired optogenetic FM decoder system in which dense and sparse decoders selectively respond to their respective signal inputs to yield distinct outputs in the form of protein interactions, gene expression, and multicellular responses. (B) A diagram highlighting the key aspects of the general signaling model (ProtoSigNet): external inputs, cell components, and reaction types. (C) A diagram depicting the multi-objective optimization strategy used to optimize for an FM decoder motif with both high performance and simplicity from a population of model candidates with initially randomized reaction parameters. (D) A scatterplot of performance vs simplicity for each model candidate in the population at generation 1, 10, or 100 aggregated from 5 optimization runs as well as the best candidates (Pareto front) across all runs. The three black shapes indicate candidates selected for further inspection. (E) Network diagrams and simulated mean output responses corresponding to the candidates chosen in (D). (F) Network diagrams and simulated mean output responses for the FM Decoder Motif with the indicated reactions ablated or modified to investigate their roles. (C, E, F) purple, orange, and grey circles represent outputs for the dense decoder, sparse decoder, or an arbitrary component, respectively. Blue lightbulb symbols represent signal inputs. Black zero symbols represent reversion processes. (E, F) Mean output was calculated as the mean of the output values during each input regime (FIG. 3B, 3C): None Input t=[0, 40), Sparse Input t=[40, 80), Dense Input t=[80, 120). (G) Simulated dense and sparse decoder mean output responses vs a range of input pulse frequencies for the FM Decoder Motif Mean output was calculated as the mean of the dynamic response during a 120 time unit span in which a given FM input is applied.

FIGS. 3A, 3B, and 3C. Dynamic responses to none/sparse/dense inputs for FM decoder candidates. (A) The ideal output response and performance target for optimization of the FM decoder motif. (B, C) Simulated dynamic responses for the corresponding motifs in FIGS. 2E and 2F.

FIGS. 4A, 4B, 4C, 4D, 4E, 4F, 4G, 4H, 4I, and 4J. Characterization of optogenetic binding dynamics using ddFP biosensors. (a, d) Diagrams of ddFP biosensor setups for characterizing the blue light dependent dynamics of the LOV/Zdk dissociation system (A) or the iLID/sspB binding system (D). (B, E) Representative fluorescence microscopy images of HEK293T cells transduced with ddFP constructs for LOV/Zdk (B) or iLID/sspB (E) before and after a 1 second pulse of 20 μW/mm² blue light exposure. (C, F) Timelapse plots showing changes in ddFP fluorescence for LOV (C) or iLID (f) reversion variants in response to a 1 second pulse of 20 μW/mm2 blue light at t=60 s compared to the plain ddFP negative control. ddFP=B3+RA; LOVfast=B3-Zdk+RA-LOVfast; LOVslow=B3-Zdk+RA-LOVslow; iLIDfast=B3-sspB+RA-iLIDfast; iLIDslow=B3-sspB+RA-iLIDslow. (G, H) Diagrams illustrating the genetic constructs and expected input response of general optogenetic implementations for the dense decoder (G) and sparse decoder (H). [A] and [B] represent any two proteins of interest. [f]=fast variant, [s]=slow variant. (I) Timelapse plot showing the ddFP fluorescence response of the sparse decoder to a none-sparse-dense sequence of blue light input regimes (20 μW/mm2). (C, F, I) Fluorescence was calculated as the mean pixel intensity of 1200×1200 pixel images taken at each timepoint and normalized as ΔF/F_0=(F_i−F_0)/F_0. Line and interval represent mean+/−1.96×SEM across n=10 timelapse repeats. (J) The mean ddFP response calculated as the mean ΔF/FO value during each of the blue light input regimes: None Input t=[0 s to 60 s), Sparse Input t=[60 s to 120 s), and Dense Input t=[120 s to 155 s). Black dots and error bars represent mean+/−1.96×SEM across 10 timelapse repeats. ns=not significant, *p<0.05, **p<0.01, ***p<0.001.

FIGS. 5A, 5B, 5C, and 5D. Measurement capabilities of the optogenetic-ddFP systems. (A) Timelapse plot showing changes in ddFP fluorescence for B3-Zdk+RA-LOVfast in response to a 1 second pulse of 20 or 200 μW/mm² blue light at t=60 s. (C) Timelapse plot showing changes in ddFP fluorescence for B3-sspB+RA-iLIDslow constructs with either 13 or 20 amino acid (AA) fusion linkers in response to a 1 second pulse of 20 μW/mm² blue light at t=60 s. (A, C) Fluorescence was calculated as the mean pixel intensity of 1200×1200 pixel images taken at each timepoint and normalized as ΔF/F_0=(F_i−F_0)/F_0. Line and interval represent mean+/−1.96×SEM across n=4 (a) or n=7 (C) timelapse repeats. (B) Minimum ΔF/F_0 value for each timelapse repeat in (a). (d) Maximum ΔF/F_0 value for each timelapse repeat in (c). (b, d) Black dots and error bars represent mean+/−±1.96×SEM across n=4 (b) or n=7 (D) time-lapse repeats. ns=not significant, *p<0.05, **p<0.01, ***p<0.001.

FIGS. 6A, 6B, 6C, 6D, 6E, 6F, 6G, and 6H. Data-driven optogenetics model recapitulates general model predictions. (a, d) Diagrams depicting the ODE models for LOV (a) or iLID (b) which describe photoconversion and dimerization, and dissociation reactions. (b-f) Simulated response of the LOV and iLID models with parameters fitted to the corresponding ddFP data (FIG. 4C, F). (G) Simulated response of a sparse decoder model (FIG. 7) formulated using the LOVfast and iLIDslow fitted parameters (b, f) compared to the Sparse-ddFP response data (FIG. 4I). (H) Simulated dense and sparse decoder mean output responses vs a range of input pulse frequencies for the previous ProtoSigNet (PSN) model (FIGS. 1 and 2) or the current OptoPI (OPI) model. Mean output was calculated as the mean of the dynamic response during a 120 time unit span in which a given FM input is applied.

FIG. 7. ODE model reactions for the optogenetic sparse decoder model. OptoPI sparse decoder model formulated using the fitted LOVfast and iLIDslow parameters (FIG. 6B, F). Equations 1-7 describe LOVfast related photoconversion and dimerization/dissociation with Zdk. Equations 8-12 describe iLIDslow related photoconversion and dimerization/dissociation with sspB.

FIGS. 8A, 8B, 8C, 8D, 8E, and 8F. FM regulation of gene transcription in HEK293T cells. (A, B) A diagram of dense and sparse decoder setups for FM regulation of red fluorescent protein (RFP) reporter gene transcription by recruiting the VP64 transcription activator to the TetR+TetO-RFP complex when exposed to the matching input. (C) RFP reporter output in response to a range of blue light input intensities administered continuously over 24 hours. (D) RFP reporter output in response to a range of blue light input pulse frequencies administered over 24 hours. (E) RFP reporter output in response to none, sparse, and dense blue light input administered over 24 hours for the TetO-RFP reporter regulated by the LOVfast or iLIDslow setups. LOVfast=TetR-LOVfast+Zdk-VP64, iLIDslow=TetR-iLIDslow+sspB-VP64. (f) FP reporter output in response to a range of blue light input pulse frequencies administered over 24 hours for the dual-FM YFP-RFP system (FIG. 9). (C, D, E, F) For all groups, reporter transcription output was calculated as the mean intensity of fluorescence microscopy images taken after 24 hours of blue light illumination and normalized to the zero/none input control group as a log 2 fold ratio. Unless denoted otherwise, blue light input intensity=10 μW/mm²; blue light pulse width=1 s. Hz=hertz (e.g 0 Hz=no light=none input, 1 Hz=continuous light=dense input, 0.25 Hz=1 s ON/3 s OFF light=sparse input). Black dots and error bars represent mean+/−1.96×SEM. ns=not significant, *p<0.05, **p<0.01, ***p<0.001.

FIG. 9. FM regulation of dual gene transcription. A diagram of the dual FM decoder system combining dense and sparse decoders for FM regulation of two genes of interest in a single cell by recruiting VP64 to either TetR+TetO-YFP or Gal4+UAS-RFP when exposed to dense or sparse input, respectively.

FIGS. 10A, 10B, 10C, 10D, 10E, and 10F. FM regulation of immune-tumor cell killing response. (a) A diagram of dense and sparse decoder setups for FM regulation of CD19 or PSMA antigen expression in K562 cells. (b) CD19 or PSMA antigen presentation as determined by antibody staining and flow cytometry. Plain K562=untransduced; Constitutive Expression=CD19 or PSMA expressed from a constitutively active SFFV promoter; Decoder=transduced with dense-CD19 or sparse-PSMA decoder constructs. Colored curves represent flow cytometry data for n=3 experimental replicates. Percentages represent the percentage of cells above the gating dashed line aggregated across all three experimental replicates. (c) CAR-T cytotoxic response towards dense-CD19 or sparse-PSMA K562 cells. Cytotoxicity was quantified by co-transducing the K562 cells with a construct expressing luciferase and measuring bioluminescence after co-culturing the K562 cells post blue light illumination with only the corresponding CAR-T cells. Black dots and error bars represent mean+/−1.96×SEM. ns=not significant, *p<0.05, **p<0.01, ***p<0.001. (d) A diagram of the bilateral tumor setup in mice with dense-CD19 and sparse-PSMA K562 tumors on the left and right flanks, respectively. I.V. injection of a mix of anti-CD19 and anti-PSMA CAR-T cells along with exposure to either dense or sparse input is expected to result in CAR-T killing of tumors carrying dense-CD19 or sparse-PSMA cells, respectively. (e) Bioluminescence imaging (BLI) signal from the bilateral tumors in mice exposed to dense or sparse input (n=5 mice for each light input group). (f) Timeseries plots of BLI signal vs days after tumor cell injection for dense-CD19 and sparse-PSMA tumors in mice exposed to dense or sparse blue light inputs. Line and error intervals represent mean+/−1.96×SEM across n=5 mice. (b, c, e, f) blue light intensity=10 μW/mm²; blue light pulse width=1 s. None input=no light=0 Hz; Dense input=continuous light=1 Hz; Sparse input=1 s ON/3 s OFF light=0.25 Hz.

FIG. 11. FM response of Dense-RFP and Sparse-RFP in K562 cells. Dense-RFP and Sparse-RFP reporter output in response to a range of blue light input pulse frequencies administered over 24 hours. For all groups, reporter transcription output was calculated as the mean intensity of fluorescence microscopy images taken after 24 hours of blue light illumination and normalized to the zero/none input control group as a log 2 fold ratio. Unless denoted otherwise, blue light input intensity=10 W/mm²; blue light pulse width=1 s. Hz=hertz (e.g 0 Hz=no light=none input, 1 Hz=continuous light=dense input, 0.25 Hz=1 s ON/3 s OFF light=sparse input). Black dots and error bars represent mean+/−1.96×SEM. ns=not significant, *p<0.05, **p<0.01, ***p<0.001.

FIGS. 12A, 12B, 12C, 12D, 12E, and 12F. FM regulation of cell differentiation fates. (A, C) A diagram of Dense-GFPlig or Sparse-mCherrylig decoder systems in K562 cells. (B, D) Representative fluorescence microscopy images of K562 cells transduced with constructs for Dense-GFPlig (a) or Sparse-mCherrylig (C) and exposed to either None, Sparse, or Dense blue light input (10 W/mm²). (E) A diagram of Dense-GFPlig or Sparse-mCherrylig sender cells and their interaction with the corresponding anti-GFPlig or anti-mCherrylig synNotch receptors on C3H receiver cells for triggering myogenic or endothelial differentiation. (F) Heatmap displaying the relative expression level of genes associated with fibroblast, endothelial, or muscular cell differentiation for dual differentiation receiver cells co-cultured with Sparse-mChlig (left) or Dense-GFPlig (right) sender cells and exposed to the corresponding input or no input. The reference column shows the corresponding results for the same receiver cell line exposed to mChlig or GFPlig, respectively, in the public dataset³³.

### DETAILED DESCRIPTION

Reference will now be made in detail to presently preferred compositions, embodiments, and methods of the present disclosure, which constitute the best modes of practicing the disclosure presently known to the inventors. The Figures are not necessarily to scale. However, it is to be understood that the disclosed embodiments are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Except in the examples, or where otherwise expressly indicated, all numerical quantities in this description indicating amounts of material or conditions of reaction and/or use are to be understood as modified by the word “about” in describing the broadest scope of the disclosure. Practice within the numerical limits stated is generally preferred. Also, unless expressly stated to the contrary: the description of a group or class of materials as suitable or preferred for a given purpose in connection with the disclosure implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this disclosure is not limited to the specific embodiments and methods described below, as specific components and/or conditions may, of course, vary. Furthermore, the terminology used herein is used only for the purpose of describing particular embodiments of the present disclosure and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e., the range from 95 to 105. Generally, when the term “about” is used, it can be expected that similar results or effects according to the disclosure can be obtained within a range of +/−5% of the indicated value.

As used herein, the term “and/or” means that either all or only one of the elements of said group may be present. For example, “A and/or B” shall mean “only A, or only B, or both A and B.” In the case of “only A,” the term also covers the possibility that B is absent, i.e., “only A, but not B.”

It is also to be understood that this disclosure is not limited to the specific embodiments and methods described below, as specific components and/or conditions may, of course, vary. Furthermore, the terminology used herein is used only for the purpose of describing particular embodiments of the present disclosure and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The phrase “composed of” means “including” or “consisting of.” Typically, this phrase is used to denote that an object is formed from a material.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5% or 10% of the value or relative characteristic.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entireties are hereby incorporated by reference into this application to more fully describe the state of the art to which this disclosure pertains.

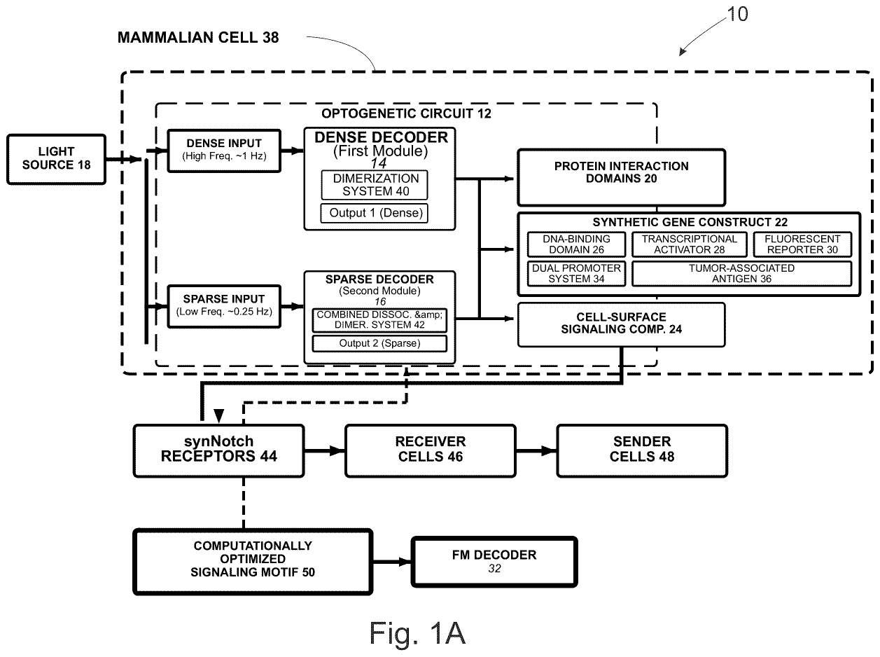

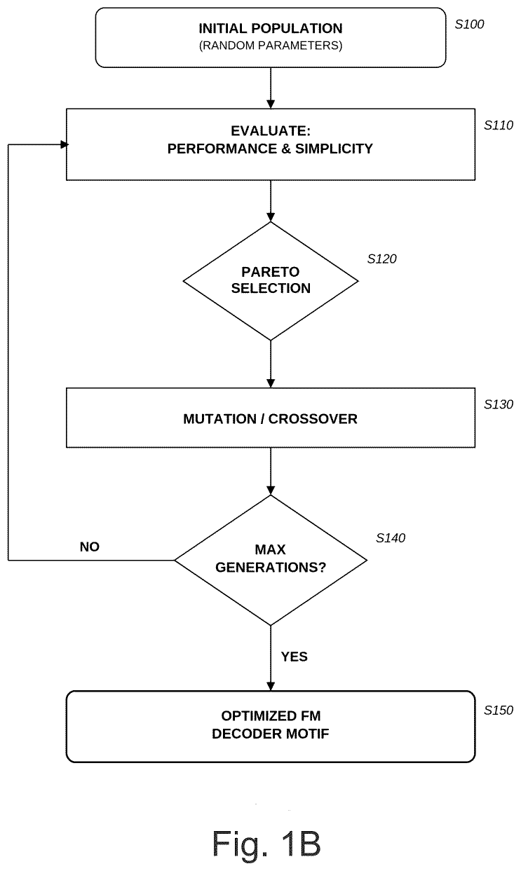

In at least one aspect, a comprehensive approach for engineering an optogenetic circuit capable of decoding frequency-modulated (FM) light inputs into multiple distinct cellular responses (see, e.g., FIG. 1a) is provided. Rather than directly regulating transcription, the system was designed to modulate protein-protein interactions, thereby expanding the scope of FM signaling applications. To realize this functionality, fluorescent biosensors were integrated with multi-objective optimization and dynamic modeling strategies to construct an FM optogenetic decoder. This methodology combines computational design with experimental validation, facilitating the prediction and refinement of system behavior across various biological contexts.

The decoder module was further integrated with additional cellular systems to modulate biological processes such as antigen presentation, demonstrating both its robustness and versatility. This streamlined framework enables the development of more advanced synthetic signaling programs and supports precise, tunable control over cellular fate decisions, representing a significant advancement in the field of cellular engineering.

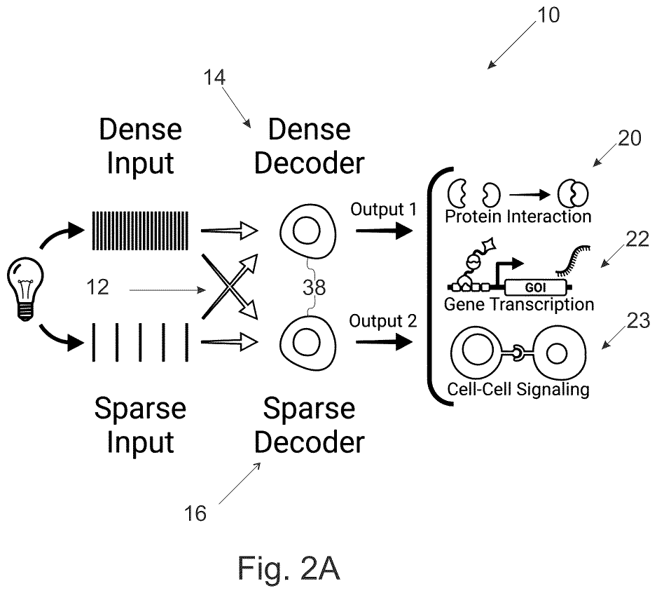

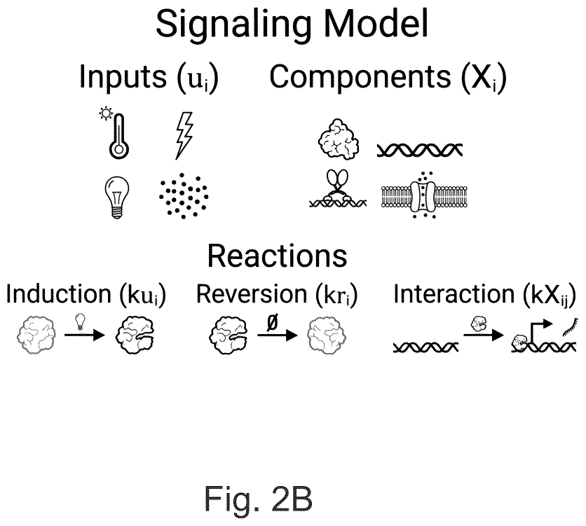

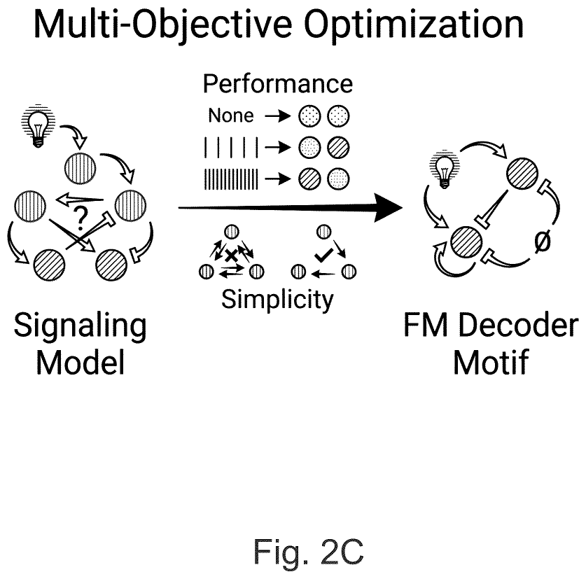

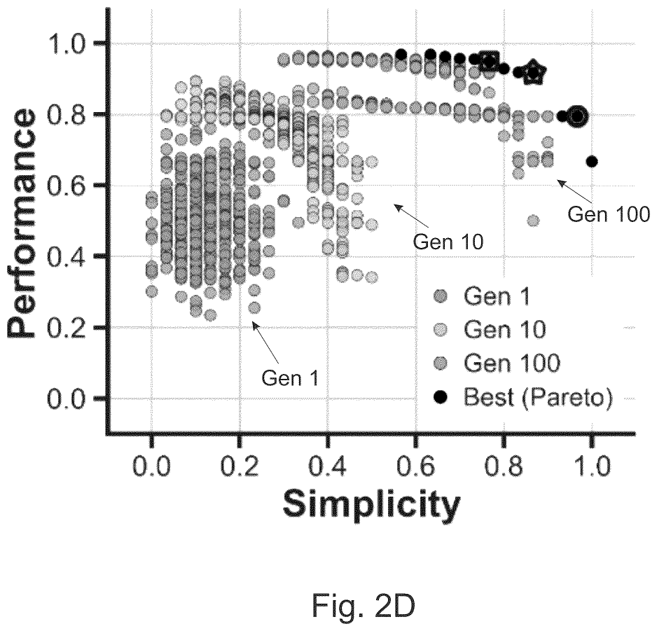

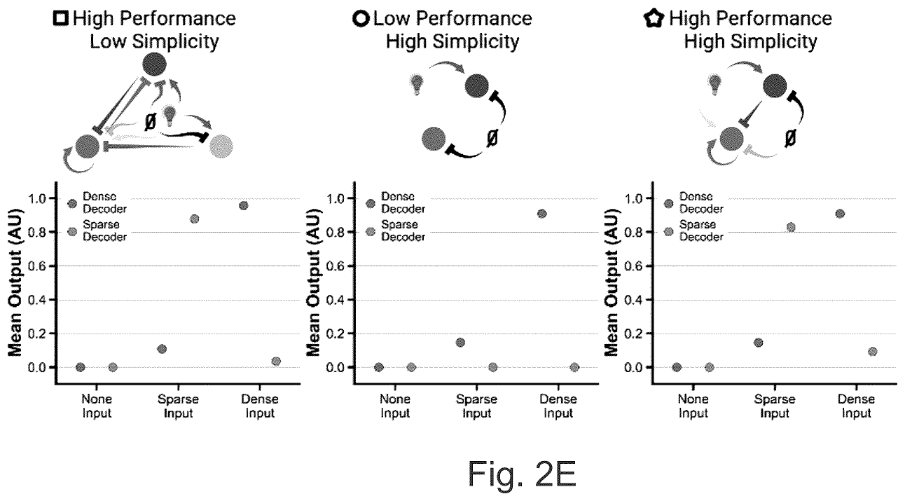

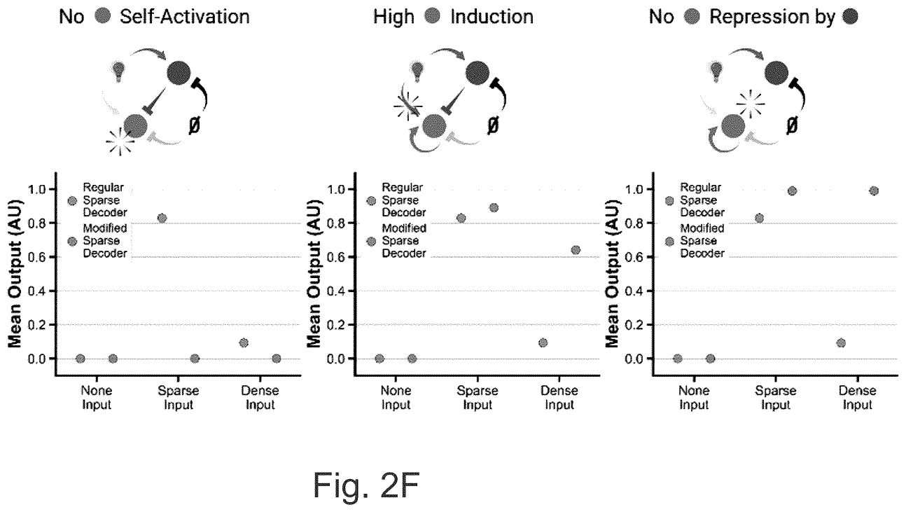

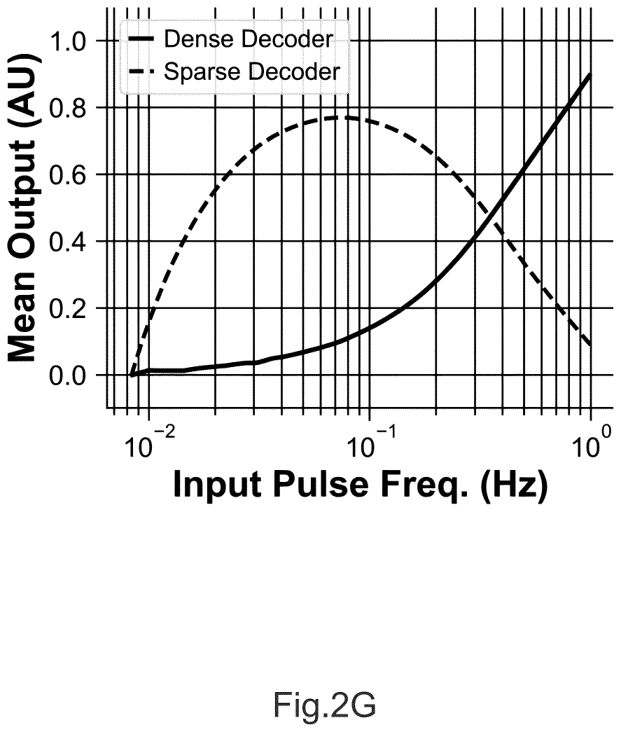

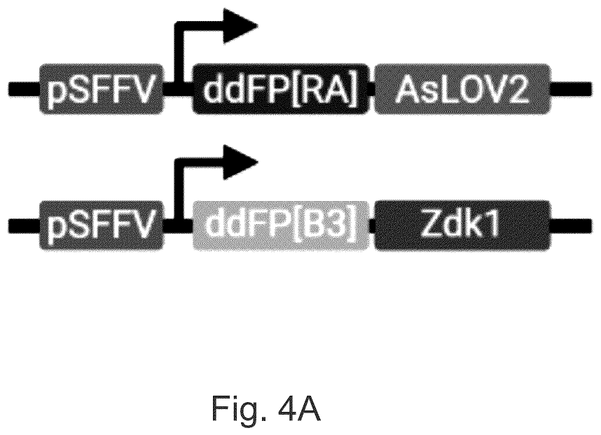

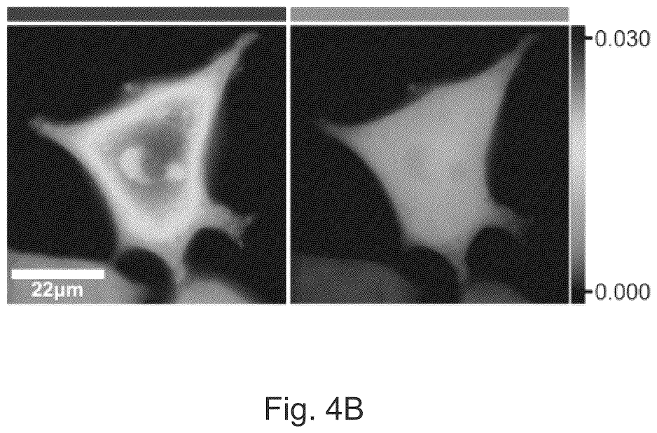

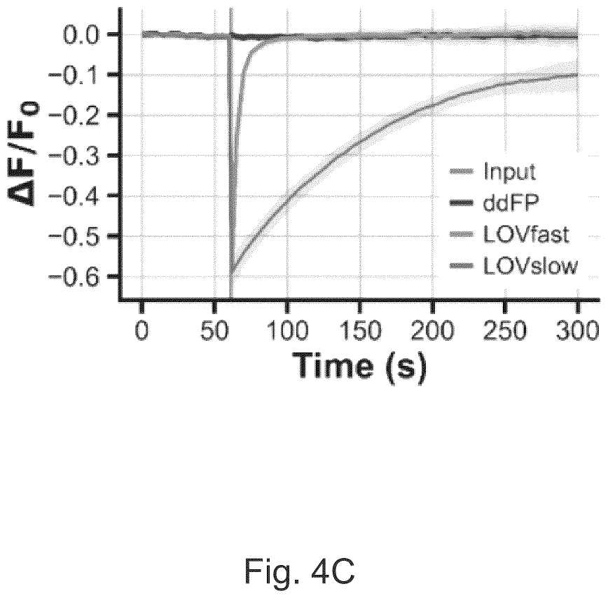

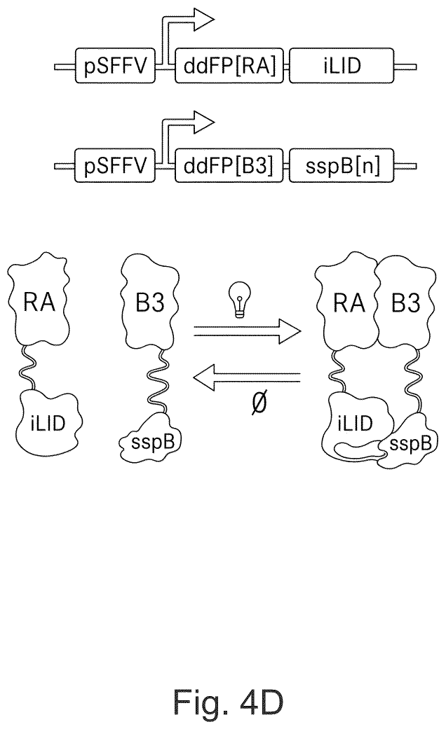

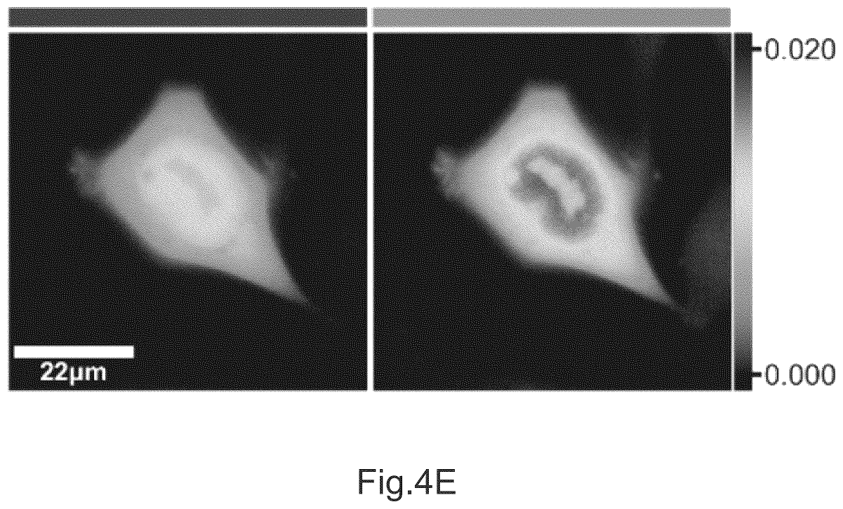

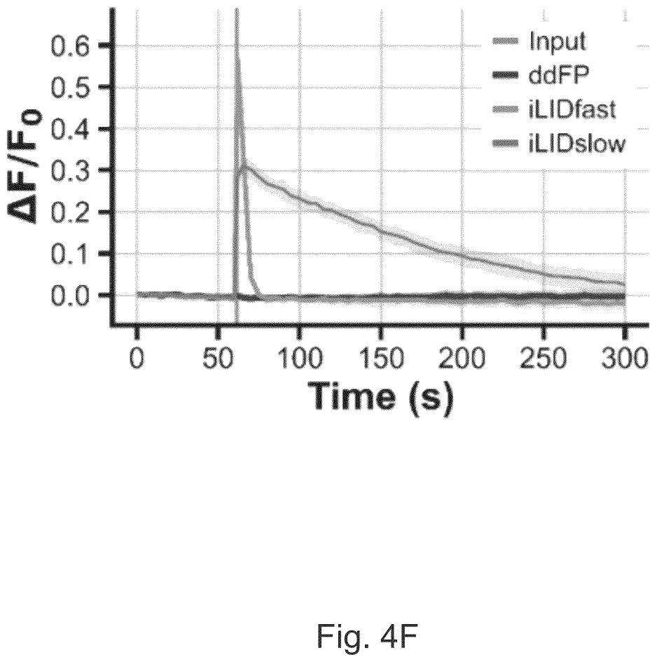

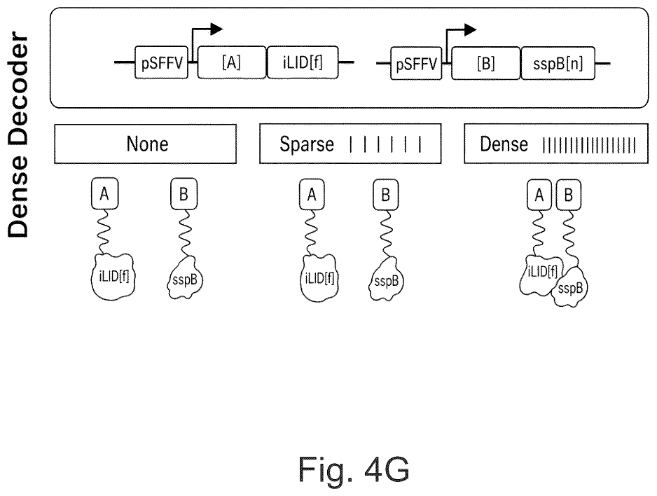

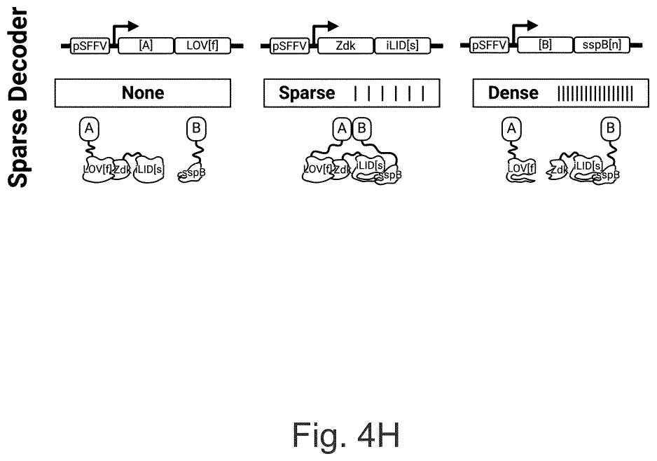

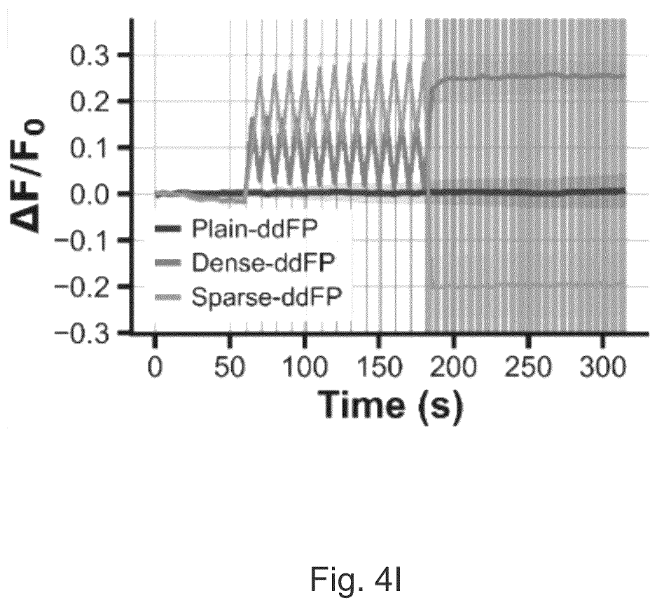

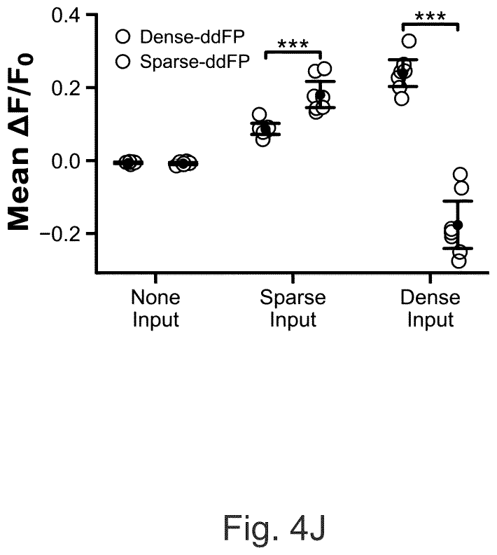

In at least one aspect, the disclosure provides a system 10 for frequency-modulated (FM) control of cellular functions in a mammalian cell 38. Referring to FIGS. 1A and 2A, this system 10 includes an optogenetic circuit 12 comprising one or both of a first light-responsive protein interaction module 14 and a second light-responsive protein interaction module 16. The first module 14 comprises a dimerization system 40 that is responsive to high-frequency blue light stimulation, herein referred to as dense input. In a refinement, the first module 14 uses iLIDfast/sspB for dimerization under high frequency “dense” input, enabling external control of biological processes (see FIG. 4G). The second module 16 includes a combined dissociation and dimerization system 42 with differential reversion kinetics, which is responsive to low-frequency blue light stimulation, referred to as sparse input. In a refinement, the second module 16 uses a combined LOVfast/Zdk and iLIDslow/sspB system with differential reversion kinetics responsive to low-frequency “sparse” input described in FIG. 4H and experimentally validated in FIG. 4I-J. The optogenetic circuit is configured to decode the frequency of the light input and to generate distinct intracellular outputs corresponding to that frequency. The cellular outputs include at least one of: (i) protein interaction domains 20 operatively linked to the optogenetic circuit 12, the protein interaction domains 20 enabling frequency-dependent control of protein-protein interactions; (ii) a synthetic gene construct 22 operatively linked to the optogenetic circuit 12, the synthetic gene construct 22 comprising one or more regulatory elements that enable selective gene transcription in response to either high-frequency or low-frequency stimulation; or (iii) a cell-surface signaling component 24 operatively linked to the optogenetic circuit 12, the cell-surface signaling component 24 enabling frequency-dependent control over cell-cell communication.

Therefore, the system 10 includes a dense decoder 14 (fast mode) and a sparse decoder 16 (slow mode) that operate based on their distinct responsiveness to frequency-modulated light input, governed by engineered differences in the reversion kinetics of their constituent proteins. Specifically, the dense decoder 14 comprises a fast-reverting photosensitive protein (iLIDfast) that achieves sustained activity only under constant or high-frequency illumination (dense input, approximately 1 Hz). Under pulsed illumination with intermittent pauses, iLIDfast rapidly reverts to its inactive state during these intervals, preventing accumulation of sufficient signaling activity. Conversely, the sparse decoder 16 integrates both a fast-reverting component (LOVfast) and a slow-reverting component (iLIDslow). This combination leverages the kinetic mismatch between these proteins: while LOVfast quickly resets during illumination pauses, iLIDslow maintains its active state for a longer duration, creating a defined temporal window during which both proteins achieve the necessary active/inactive configuration under pulsed (sparse) illumination conditions (~0.25 Hz). Consequently, these two decoder systems exhibit complementary, mutually exclusive activation profiles, such that the dense decoder 14 preferentially activates during constant or high-frequency light input, whereas the sparse decoder 16 activates primarily under intermittent or lower-frequency conditions. This complementary relationship was precisely optimized through computational modeling, enabling a single light wavelength to independently and selectively control two distinct cellular functions solely by modulating the frequency and pattern of illumination, analogous to frequency-based tuning in radio communications.

In a further aspect, the second light-responsive protein interaction module 16 may be characterized as comprising a fast-reverting dissociation component and a slow-reverting dimerization component. The fast-reverting dissociation component comprises a LOVfast/Zdk pair that rapidly dissociates upon blue light exposure and quickly re-associates during dark intervals. The slow-reverting dimerization component comprises an iLIDslow/sspB pair that dimerizes upon blue light exposure and maintains the dimerized state for a substantially longer duration after illumination ceases. The kinetic mismatch between the reversion rates of the fast-reverting dissociation component and the slow-reverting dimerization component creates a temporal window during pulsed illumination in which both components simultaneously achieve a signaling-active configuration. During this temporal window, the LOVfast/Zdk pair has re-associated (because its fast reversion allows rapid recovery) while the iLIDslow/sspB pair remains dimerized (because its slow reversion maintains the active state). This concurrent signaling-active configuration of both components enables the sparse decoder 16 to generate its output selectively in response to low-frequency pulsed illumination.

In the context of the disclosed frequency-modulated optogenetic system, the terms “dense” and “sparse” specifically refer to distinct temporal patterns of illumination applied to the engineered cells. The “dense” pattern is characterized by high-frequency stimulation (approximately 1 Hz), consisting of continuous or rapidly pulsed illumination with minimal or no intervals between successive pulses, resulting in a condition wherein illumination is effectively sustained or continuously “on.” This dense illumination optimally activates the dense decoder components. Conversely, the “sparse” pattern involves low-frequency illumination (approximately 0.25 Hz), comprising discrete pulses separated by substantial intervals of darkness, such that the illumination is “off” for longer durations relative to the “on” periods. This sparse illumination selectively activates the sparse decoder components. The distinction between dense and sparse illumination thus reflects the temporal density of pulses within a given interval: dense illumination being continuous or closely spaced pulses, and sparse illumination characterized by pulses spaced farther apart in time. The optogenetic system described herein is intentionally engineered with separate decoder systems to enable selective and independent cellular responses based solely on these differing illumination frequency patterns.

In another aspect, the system may be configured to incorporate either a single decoder (dense or sparse) or both decoders simultaneously, depending on the intended application. In single-decoder configurations, either the dense decoder 14 (comprising iLIDfast and sspB components) or the sparse decoder 16 (comprising LOVfast/Zdk and iLIDslow/sspB components) can be independently implemented, as demonstrated experimentally. Alternatively, in a dual-decoder configuration, both dense and sparse decoders 14, 16 are integrated within the same cell. This dual implementation was exemplified through a gene transcription experiment wherein the dense decoder regulated expression of a gene under control of the TetR/TetO system (mGold), while the sparse decoder regulated expression of a separate gene under control of the Gal4/UAS system (mScarlet). Significantly, this dual-decoder configuration enabled independent, multiplexed control of distinct genes within the same cell by solely modulating the frequency pattern of a single light input. This represents an important advancement by providing multiplexed intracellular signaling without the need for additional wavelengths of light or secondary regulatory mechanisms.

In another aspect, the system 10 includes a computationally optimized signaling motif 50 that serves as the optogenetic circuit 12 and is configured to decode the frequency of light input to yield discrete intracellular outcomes. In this regard, an optimization algorithm is applied to the signaling model to identify a specific set of parameter values that would yield a simple network motif with FM decoding behaviors. The signaling motif can include a computationally evolved network of ordinary differential equations that has been optimized using a multi-objective evolutionary algorithm, such as the Non-dominated Sorting Genetic Algorithm II (NSGA-II), in order to balance simplicity and FM decoding fidelity.

In another aspect, the first light-responsive protein interaction module 14 may specifically include an iLID/sspB protein pair (FIG. 4G), wherein dimerization occurs under continuous or high-frequency blue light. The second light-responsive protein interaction module 16 may include an LOV/Zdk pair that dissociates under continuous or high-frequency light stimulation and re-associates during sparse or absent light conditions due to slower reversion kinetics.

In another aspect, the system 10 further includes a synthetic gene construct 22 including a DNA-binding domain 26, such as TetR or Gal4, that is operatively linked to a light-inducible transcriptional activator 28, such as VP64. In certain configurations, the synthetic gene construct 22 includes a dual promoter system 34 configured to independently drive expression of a first gene in response to dense input and a second gene in response to sparse input. For example, the experiments below detail a dual FM system for controlling the expression of two target genes simultaneously within the same cell in which TetR/TetO in the sparse decoder system is substituted with Gal4/UAS to obtain independent transcriptional readouts for each decoder.

In a refinement, the DNA-binding domain 26 is operatively linked to a light-inducible transcriptional activator 28. In this configuration, frequency-dependent dimerization or dissociation of the optogenetic circuit 12 recruits the transcriptional activator 28 to the DNA-binding domain 26 to drive expression of a gene of interest. For example, in the dense decoder configuration, blue light at a dense input frequency causes iLIDfast-mediated dimerization that recruits sspB-VP64 to TetR, which in turn drives expression of the downstream reporter or target gene. In the sparse decoder configuration, the combined LOVfast/Zdk dissociation and iLIDslow/sspB dimerization events under sparse input conditions similarly recruit VP64 to the DNA-binding domain to drive expression of a gene of interest under the cognate promoter.

In another aspect, to facilitate visualization and quantitative assessment, the system 10 may also include a fluorescent reporter construct 30 configured such that frequency-modulated gene expression results in the detectable expression of at least two different fluorescent proteins, each corresponding to a different frequency regime of light input (e.g., corresponding to dense and sparse light inputs). For examples, two fluorescent reporter constructs, TetO-mGold and UAS-mScarlet demonstrated frequency selective behavior in response to different light inputs.

In another aspect, the synthetic gene construct 22 encodes a tumor-associated antigen 36, such as CD19 or PSMA, that is presented on the cell membrane in a frequency-dependent manner (i.e., frequency-dependent recognition), enabling selective recognition and targeting by chimeric antigen receptor (CAR) T cells. Alternatively, the cell-surface signaling component 24 enables paracrine signaling that induces differentiation in co-cultured receiver cells through frequency-dependent expression of synthetic ligands. In this regard, either Dense-GFP_lig or Sparse-mCh_lig sender cells were co-cultured with C3H dual differentiation receiver cells, which possess receptors that regulate the expression of MyoD and ETV2, thereby driving differentiation into muscle-like or endothelial-like cells, respectively.

In another aspect, the system may be adapted such that these ligands engage engineered synNotch receptors 44 present on co-cultured receiver cells 46, thereby inducing cell fate transitions such as differentiation into myogenic or endothelial lineages.

In another aspect, the method of using the system 10 may include contacting the mammalian cell with chimeric antigen receptor (CAR) T cells that selectively target a tumor-associated antigen expressed on the cell membrane of the mammalian cell in a frequency-dependent manner. For example, K562 cells engineered with the dense decoder to express CD19 on the cell membrane upon dense input, or with the sparse decoder to express PSMA on the cell membrane upon sparse input, may be contacted with anti-CD19 or anti-PSMA CAR T cells, respectively, to elicit a frequency-selective cytotoxic immune response. This contacting step enables selective targeting of tumor cells based on which antigen has been induced by the corresponding light input frequency.

In another aspect, the optogenetic circuit 12 used in the system 10 may be configured as an FM decoder 32 that exhibits differential output across a range of light input frequencies, typically within the range of 0.05 Hz to 1 Hz. The FM decoder 32 may further be adapted to enable multiplexed gene expression control in a single mammalian cell, thereby supporting complex synthetic logic operations and combinatorial outputs.

In summary, the computationally optimized signaling motif 50 underlying the optogenetic circuit 12 comprises a computationally evolved network of ordinary differential equations that has been optimized to balance simplicity and FM decoding fidelity. The optogenetic circuit 12, configured as an FM decoder 32, exhibits differential output across a range of light input frequencies and enables multiplexed control of gene expression in a single mammalian cell. The system 10 generates distinct intracellular outputs in response to different light input frequencies, including protein interaction domains 20 enabling frequency-dependent control of protein-protein interactions. The cell-surface signaling component 24 enabling frequency-dependent control over cell-cell communication provides an additional output modality. Each of these distinct intracellular outputs may be independently and selectively activated by modulating the frequency of a single blue light source.

In another aspect, the system 10 described herein may be implemented in mammalian cell lines such as HEK293T, K562 tumor cells, or C3H fibroblast cells, and may be used in vitro or in vivo for applications in basic research, therapeutic engineering, or regenerative medicine.

The following examples illustrate the various embodiments of the present disclosure. Those skilled in the art will recognize many variations that are within the spirit of the present disclosure and scope of the claims.

### Results

### General Model Representation of Synthetic Signaling Networks

To determine a conceptual design for an FM decoder system, an ordinary differential equation (ODE) model for prototyping signaling networks (ProtoSigNet) is formulated, which can be optimized towards desired dynamic behaviors and other user-defined objectives (FIG. 2B). This simple model broadly represents the dynamics of cell signaling components and how their states are influenced by reactions from external inputs and other components in the system. Cell components (Xᵢ) are defined as elements such as optogenetic proteins, enzymes, or other gene products which can affect each other through signaling reactions. On the other hand, stimuli inputs (uᵢ) represent signals such as light, heat, or ligands which can affect the state of cell components but are themselves unaffected by other components or inputs.

For simplicity, three general categories of signaling reactions are defined in the model: baseline reversion, stimuli induction, and component interaction. These reactions serve as driving forces that shift a component towards low (Xᵢ=0) or high (Xᵢ=1) activity states. Reversion reactions (krᵢ) represent the resting tendency of each component for either of these states (e.g., an optogenetic protein that is considered mainly in its closed or open conformation at rest). Induction reactions (kuᵢ) represent the influence of external stimuli inputs towards each component (e.g., light converting an optogenetic protein into its open conformation). Lastly, the interaction reactions (kXᵢⱼ) govern how each component j affects the state of each component i (e.g., a transcription factor's influence on the production of some target protein).

### Multi-Objective Optimization of an FM Decoder Motif

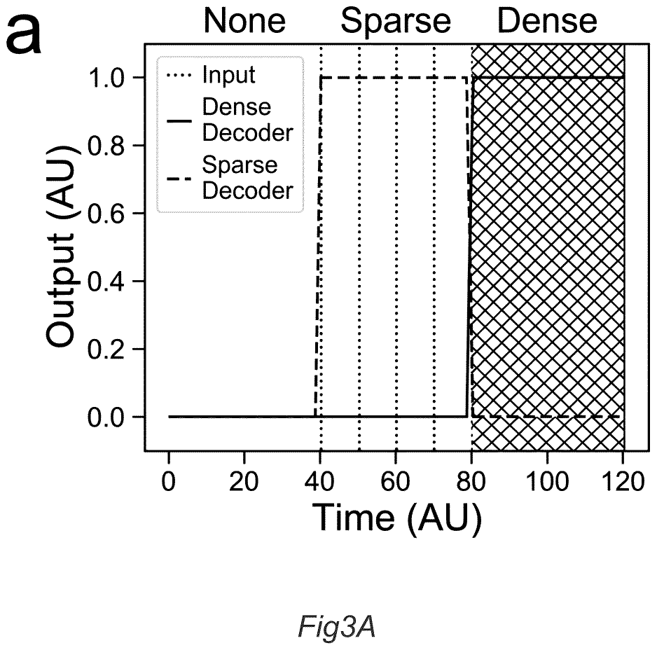

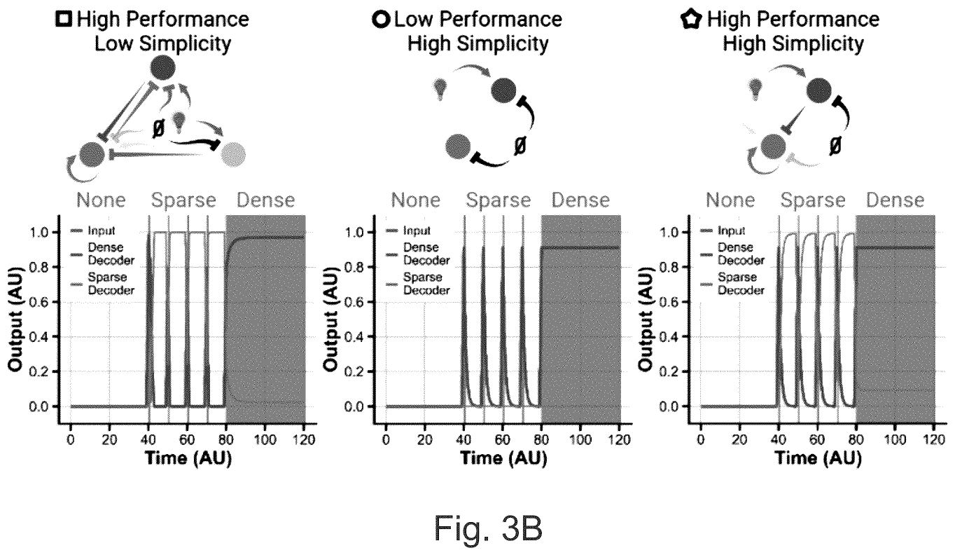

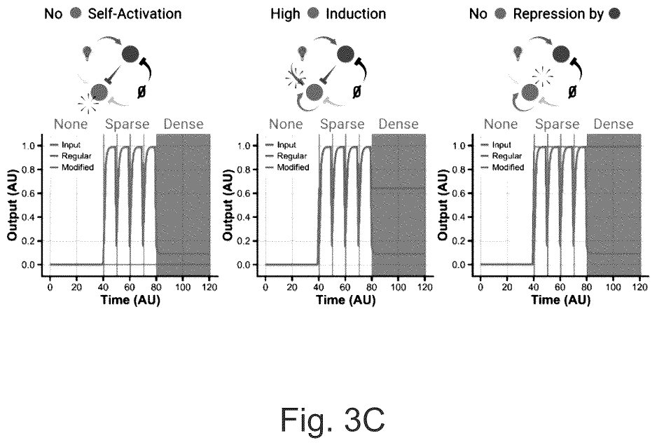

An optimization algorithm was applied to the signaling model to identify a specific set of parameter values that would yield a simple network motif exhibiting FM decoding behaviors (FIG. 2C). As shown in FIG. 2B, the optimization procedure comprises the following steps. This optimization framework was designed to maximize two objectives: performance and simplicity. Model performance was defined by high output of one component (X1, purple node) exclusively in response to a high-frequency (dense) input signal, and high output of a second component (X2, orange node) solely in response to a low-frequency (sparse) input signal (FIG. 3A). Simplicity was promoted by minimizing the number of reactions in the model, discouraging overly complex designs that, while potentially high performing, would be less feasible for implementation in mammalian cell systems.

To carry out the numerical optimization, the Non-dominated Sorting Genetic Algorithm II (NSGA-II)⁽¹⁹⁾, a well-established multi-objective optimization algorithm, was employed. NSGA-II evolves a population of model candidates/individuals (S100) through a process of objective scoring, parent selection, mating, and survivor selection to enrich for higher fitness offspring over many generations. By applying the algorithm to the signaling model and specified objectives (S110), population-wide improvements in both performance and simplicity were observed across successive generations of the optimization process (FIG. 2D). In the initial generation, the population exhibited a wide distribution in objective space, indicative of their quasi-randomized starting parameters. By the 10th generation, however, the population had begun to converge toward an optimality front, with further refinements observed by the 100th generation. Given the stochastic nature of the algorithm, the optimization procedure was repeated, and the top-performing candidates from five independent runs were plotted, collectively forming a Pareto front (S120)²⁰.

Since multi-objective optimization results in multiple viable solutions along the Pareto front, it was necessary to select a single motif for implementation. Three candidates exhibiting different trade-offs between performance and simplicity were selected from the Pareto front for further examination. Through successive rounds of mutation and crossover (S130), the algorithm iterates until a maximum number of generations is reached (S140). Network diagrams were generated, and output responses were simulated based on their respective parameter sets (FIG. 3B). To assess the performance of each candidate, the mean output of the dense or sparse decoder during the corresponding input regimes was calculated (FIG. 2E). The high-performance/low-simplicity candidate involved a large number of reactions, making it impractical for implementation. Conversely, the low-performance/high-simplicity candidate failed to exhibit the desired frequency-modulated (FM) decoding behavior. The third candidate, although not as extreme in either performance or simplicity, offered a balanced trade-off and was selected as the Frequency Modulated Decoder Motif (FMDM) (S150) for subsequent analysis.

The FMDM (FIG. 3B, right side) consists of two components corresponding to the dense (purple) and sparse (orange) decoder output states. The dense decoder component is affected by a strong induction response to stimuli input and a fast reversion reaction. In response to sparse input, the dense decoder output would spike but then decay rapidly resulting in low levels on average. However, exposure to dense input would be sufficient to sustain its output at high levels. For the sparse decoder component there are four reactions involved. Under dense input, the sustained strong repression interaction from the dense decoder component results in low levels of sparse decoder output since the repression strength exceeds the weaker induction reaction on the sparse decoder component. However, under sparse input, the repression interaction is not sustained. This allows the effect of the weak induction, slow reversion, and self-activation reactions to increase the sparse decoder output in between the stimuli pulses resulting in high output levels on average.

The roles of specific reactions contributing to the sparse decoder state in the Frequency Modulated Decoder Motif (FMDM) were further investigated by altering their magnitudes. The sparse decoder output of the original model was compared to that of the modified model through simulation (FIG. 3C), and the mean output during each input regime was calculated (FIG. 2F). Ablating the self-activation reaction resulted in lowered sparse decoder output across all input regimes. This demonstrated that the reaction is important for achieving high output levels during sparse input as it allows the sparse decoder component to build up in between input pulses. Increasing the strength of the induction reaction on the sparse decoder component resulted in higher output levels during dense input which underscores how the sparse decoder output is a balance between the induction and repression reactions during this regime. Lastly, ablating the repression reaction also led to high output levels during dense input which also reinforces the same notion.

Although the original optimization was conducted using fixed pulse frequencies—1 Hz for dense inputs and 0.1 Hz for sparse inputs—the Frequency Modulated Decoder Motif (FMDM) was subsequently employed to predict the responses of the dense and sparse decoders across a broader range of input pulse frequencies (FIG. 2G). For the dense decoder, its average output increased monotonically with increasing input frequency, peaking at the maximum 1 Hz (equivalent to a continuous input signal). On the other hand, the sparse decoder average output peaked at approximately 0.07 Hz before decreasing with higher input.

### Biosensor Characterization of Optogenetic Protein Interactions

To facilitate implementation of the Frequency Modulated Decoder Motif (FMDM) in mammalian cells, a search was conducted for optogenetic protein components possessing dynamic properties and signaling features that aligned with the motif's functional requirements. One key requirement was having components with differential reversion rates (krᵢ) and induction rates (kuᵢ). This led us to explore the blue light responsive dimerization and dissociation systems, iLID-sspB²¹ and LOV-Zdk²² which have well-documented mutations that change their induction and reversion dynamics^{23,24}.

Utilizing the RA and B3 ddFP biosensor domains⁽²⁵⁾, the dynamic responses of iLID and LOV to blue light stimulation were monitored in HEK293T cells. To examine the blue light-inducible dissociation between LOV and Zdk, fusion constructs RA-LOV and B3-Zdk were generated (FIG. 4A). Following co-transduction of HEK293T cells with these constructs, a 1-second pulse of blue light was applied, resulting in a decrease in ddFP fluorescence, indicative of dissociation between LOV and Zdk (FIG. 4B). In contrast, an increase in ddFP fluorescence was observed under identical conditions when using the fusion constructs RA-iLID and B3-sspB, consistent with blue light-induced binding between iLID and sspB (FIGS. 4D-E). Given that the Frequency Modulated Decoder Motif (FMDM) required components exhibiting both relatively fast and slow reversion rates, the dynamics of two widely used reversion variants were profiled: the I427V fast reversion variant (iLIDfast/LOVfast) and the V416I slow reversion variant (iLIDslow/LOVslow). By tracking the binding dynamics of these LOV and iLID variants over time using ddFP fluorescence and microscopy (FIGS. 4C and 4F), it was observed that all groups maintained stable fluorescence prior to blue light exposure at t=60 seconds. Upon exposure, the ddFP control group (plain B3 and RA) showed no significant deviation from baseline. However, LOVfast and LOVslow displayed a significant decrease, while iLIDfast and iLIDslow exhibited a significant increase in fluorescence at their respective induction peaks, relative to baseline.

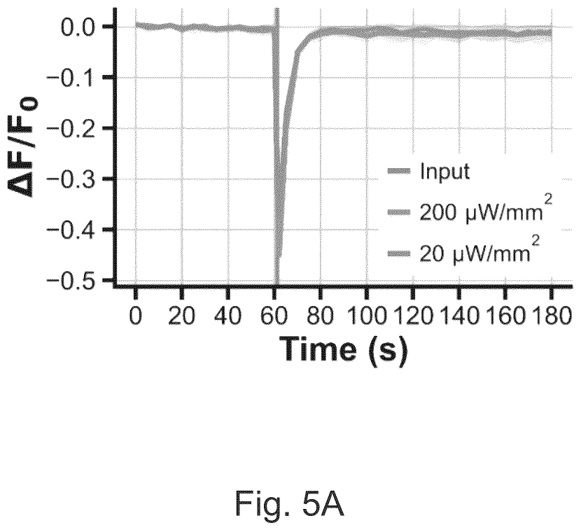

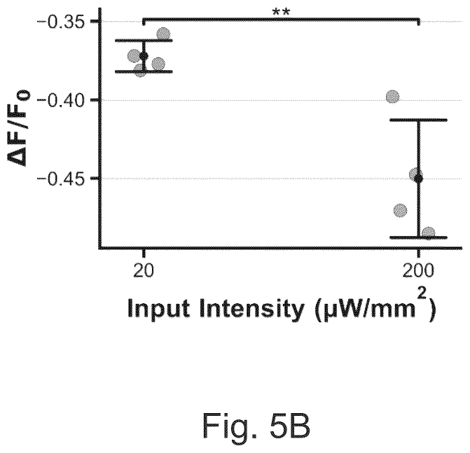

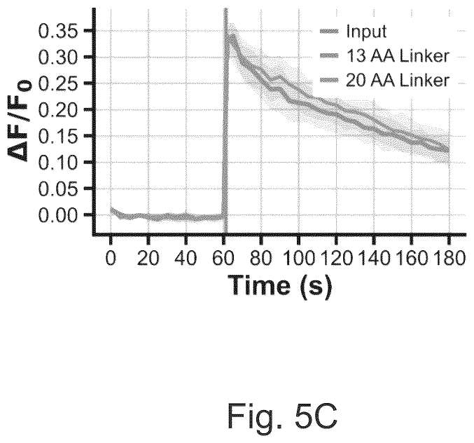

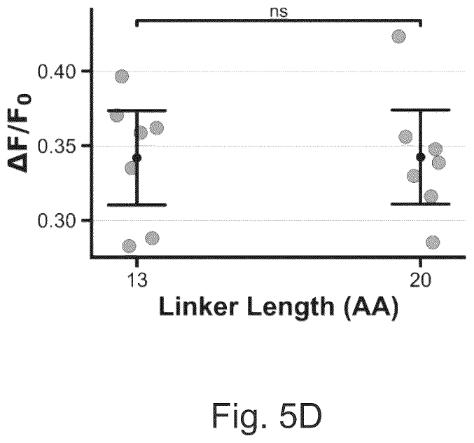

Additional tests to explore the measurement capabilities of ddFP were conducted. The LOVfast-ddFP system was subjected to a is blue light pulse of either 20 μW/mm² or 200 μW/mm² intensity and the response over time was tracked using fluorescence microscopy (FIG. 5A). Examining the response after the blue light pulse revealed that the higher intensity resulted in a significantly lower decrease in fluorescence (FIG. 5B), potentially reflecting the greater LOV-Zdk dissociation. On a similar note, the iLIDslow variant appeared to have a lower magnitude of induction compared to the other optogenetic components (FIG. 4F). The investigation was extended by varying the length of the fusion linkers between RA/B3 and iLIDslow/sspB. Subjecting these constructs to a is pulse of 20 μW/mm² blue light resulted in no significance in dynamic response (FIG. 5C) and peak fluorescence (FIG. 5D). While other factors could contribute to the magnitude of ddFP change, these results suggest that iLIDslow has a weaker induction change compared to LOVfast. This meant that iLIDslow has the potential to fulfill the role of the weak induction reaction towards the sparse decoder component in the FMDM.

The remaining FMDM requirements include the ability of one component to strongly repress the output of another component and the ability of a component to recover or build up output levels in between input pulses. For the first feature, it was noted that LOV and iLID exert opposing effects towards protein interactions (dissociation vs dimerization) and the dissociation effect of LOV could potentially be used to repress the dimerization effect of iLID. Furthermore, by combining the LOVfast and iLIDslow variants in particular, the second feature could also be mediated. Due to the slow reversion rate of iLIDslow, iLIDslow-sspB binding should remain high in between each input pulse. Meanwhile, the fast reversion of LOVfast-Zdk dissociation should allow the overall output of the sparse decoder to rapidly recover in between each input pulse, fulfilling the general effect of the self-activation reaction.

### FM Control of Light Inducible Protein Interactions

Based on the characterization results for LOV and iLID, a cellular signaling circuit for controlling protein interactions via FM blue light inputs was assembled. Given two proteins or elements, [A] and [B], that produce a cellular response in proportion to their interactions, the dense decoder can be implemented as the fusion constructs, iLIDfast-[A] and sspB-[B](FIG. 4G). Similarly, the sparse decoder can be implemented through LOVfast-[A], Zdk-iLIDslow, and sspB-[B](FIG. 4H). During none input, both dense and sparse decoders can be expected to have low average output due to the lack of binding between both iLIDfast/sspB and iLIDslow/sspB. During sparse input, the light stimulation should cause the iLIDslow/sspB pair to engage and the LOVfast/Zdk pair to disengage. However, the different reversion rate respective to each pair creates a window where both pairs are bound and active, leading to high average output for the sparse decoder. In contrast, the fast reversion dynamics of iLIDfast should lead to iLIDfast/sspB favoring the unbound state, resulting in low output for the dense decoder. Lastly, dense input is expected to maintain iLIDfast/sspB in the bound state and LOVfast/Zdk in the unbound state leading to high dense decoder and low sparse decoder output, respectively.

To validate the expected function of the dense and sparse decoders, HEK293T cells were transduced with either Dense-ddFP or Sparse-ddFP constructs, in which the [A] and [B] domains correspond to RA and B3 and exposed the cells to a none-sparse-dense sequence of FM light input. The dynamics of the ddFP response quantified from fluorescence timelapse images showed the expected FM decoding behavior (FIG. 4I, consistent with the previous simulation results (FIG. 3B, right side). The mean fluorescence response of the system was also quantified under conditions of no input, sparse input, and dense input. A significant difference was observed between the mean outputs of each decoder when comparing the sparse and dense input regimes (FIG. 4J). These results confirmed the successful implementation of the FMDM as an optogenetic system for regulating protein interactions in mammalian cells.

### Optogenetic Protein Interactions Model Predicts FM Responses

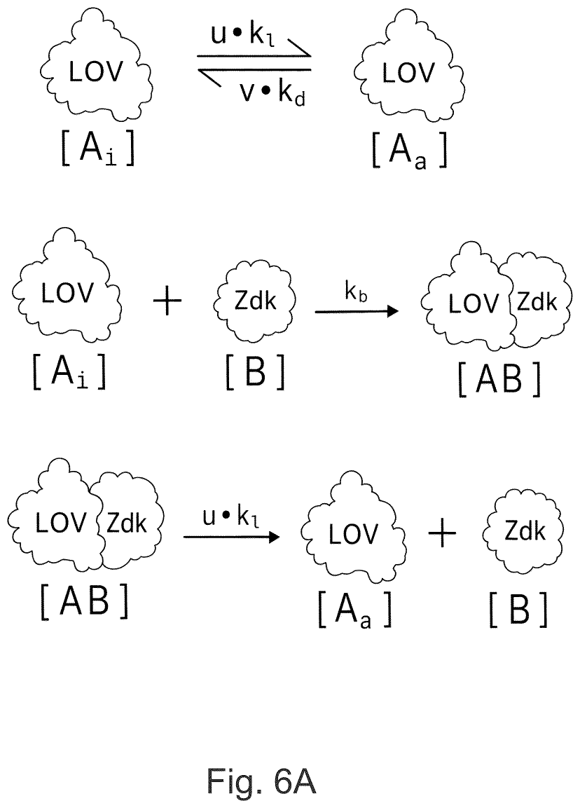

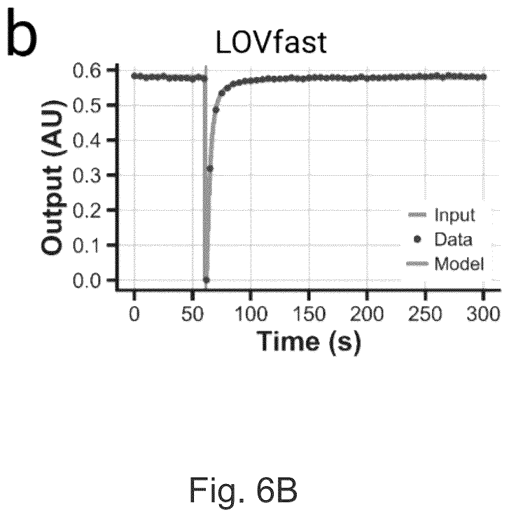

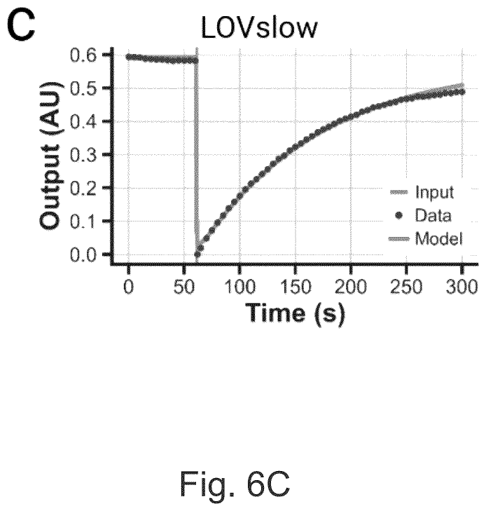

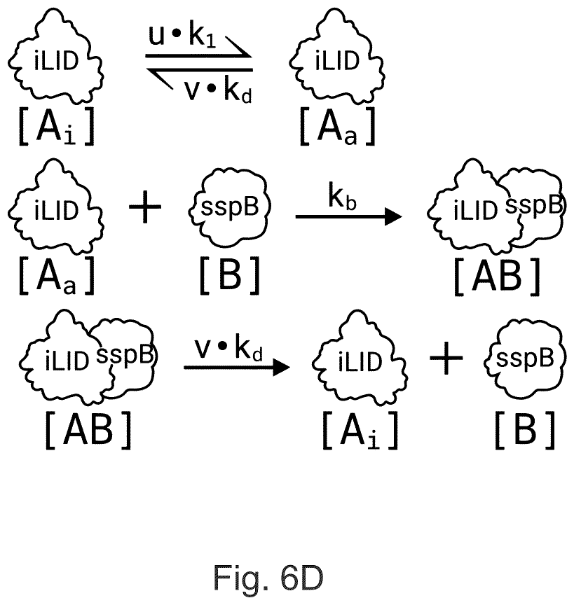

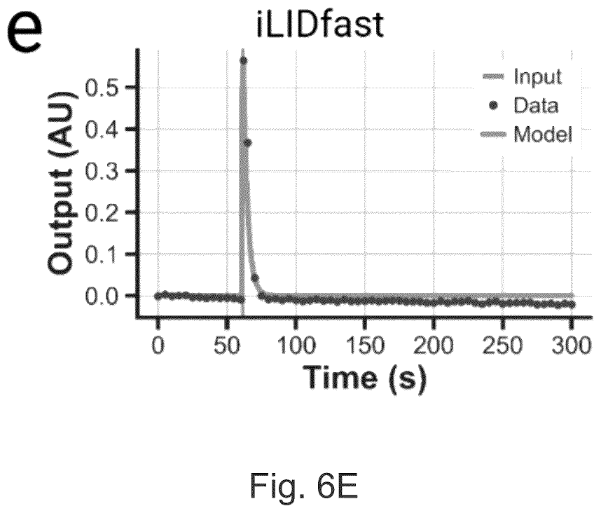

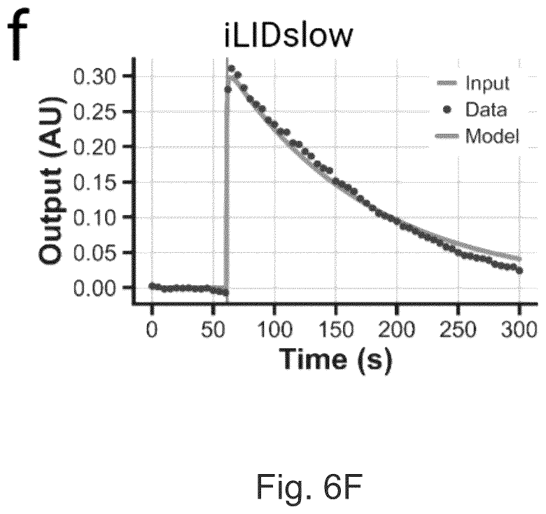

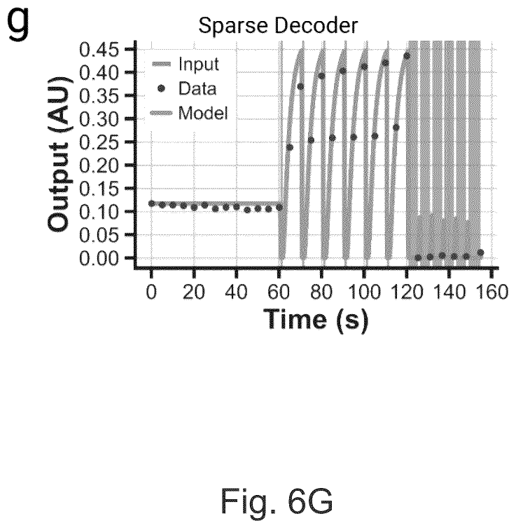

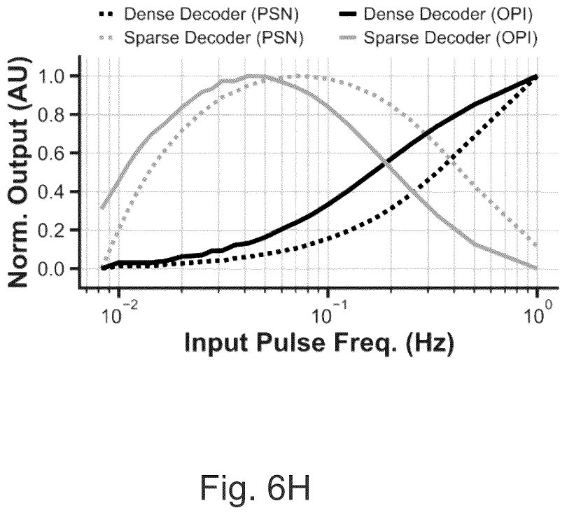

To further utilize the ddFP data, an ordinary differential equation (ODE) model was formulated to predict the responses of the optogenetic frequency-modulated (FM) decoders. In contrast to the ProtoSigNet model, which was a general signaling model for high level prototyping, this optogenetic protein interactions (OptoPI) model is tailored to the specific reaction mechanisms of LOV/Zdk (FIG. 5A) and iLID/sspB (FIG. 6D). In this model, both LOV and iLID photoconvert towards the open/active (Aₐ) state in the presence of light input and revert towards the closed/inactive (Aᵢ) state in its absence. Two other reactions represent LOV or iLID interaction with their respective binding partners (B) to form a complex (AB), which is then able to dissociate back into the individual components. The main difference is that LOV binds to Zdk in its inactive state and dissociates when activated, while it is the opposite for iLID/sspB. To estimate the parameter values for the reaction rates, the model response of the bound state (AB) was fitted to the previously obtained ddFP data for LOV and iLID (FIGS. 6B, 6C, 6E, and 6F).

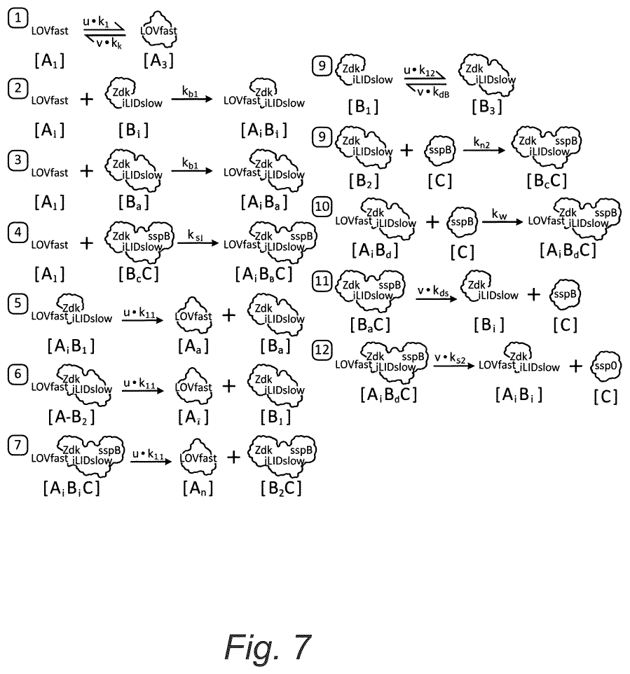

The estimated parameter values were subsequently incorporated into ODE models for the dense and sparse optogenetic decoders. While the dense decoder consisted solely of the iLIDfast/sspB pair, an ordinary differential equation (ODE) model was constructed for the sparse decoder to represent its dual-component architecture, incorporating both LOVfast/Zdk and iLIDslow/sspB interactions (FIG. 7). Upon simulating this model with the corresponding parameter values, the dynamic response of the complete complex—where both LOVfast/Zdk and iLIDslow/sspB are simultaneously bound—closely aligned with the previously acquired Sparse-ddFP data (FIG. 6G). This result demonstrated that the circuit's response could be effectively predicted using the individually fitted models of its component parts.

Similar to the ProtoSigNet model, the OptoPI model was used to simulate the mean output for a range of input pulse frequencies, allowing us to predict the frequency response of the optogenetic FM decoders (FIG. 6H). The frequency response curves of the ProtoSigNet and OptoPI models were compared, revealing similar trends in the behavior of both the dense and sparse decoders. Since the ProtoSigNet model was formulated to be a general signaling model with coarse-grained parameter values, it was possible for the behavior of the optogenetic implementation to highly deviate from the design. However, the results here showed that the optogenetic components and circuit implementation were able to fulfill the signaling requirements of the FMDM and recapitulate its simulated behaviors.

### FM Control of Light Inducible Gene Transcription

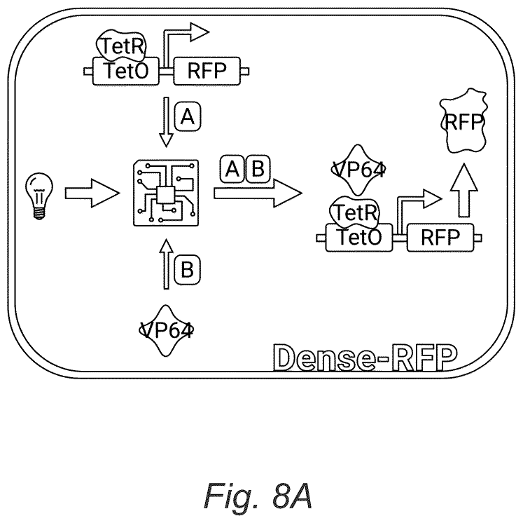

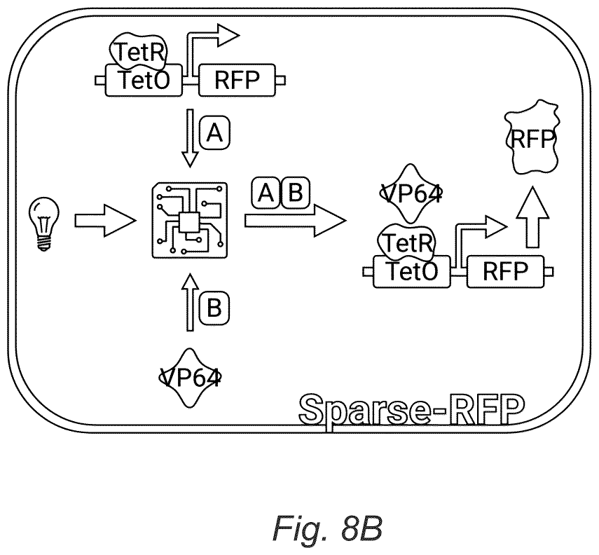

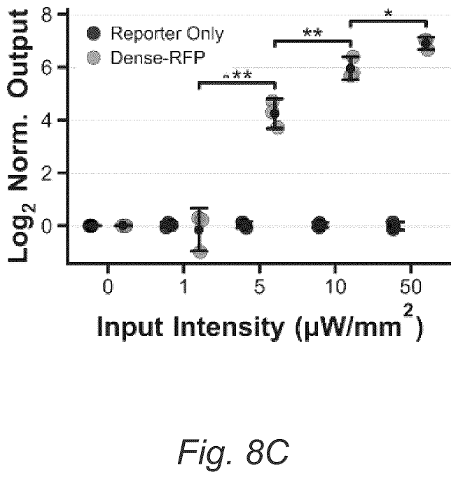

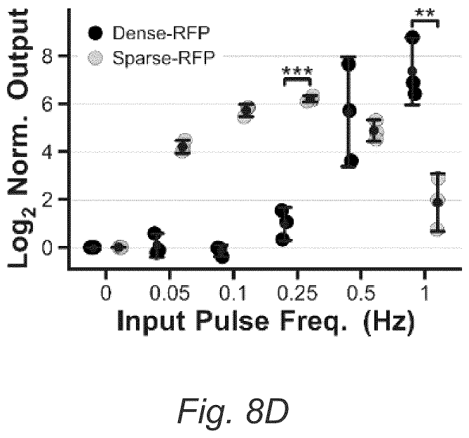

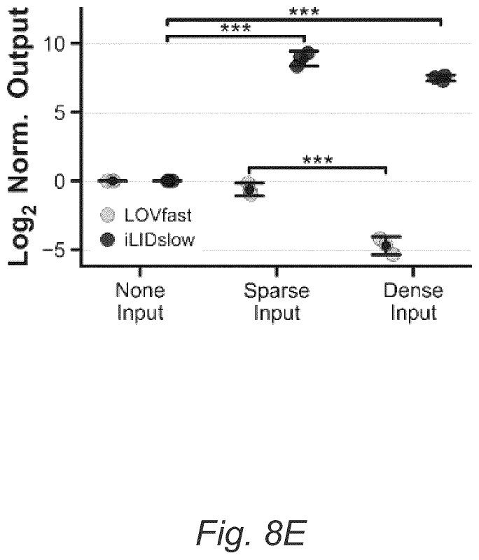

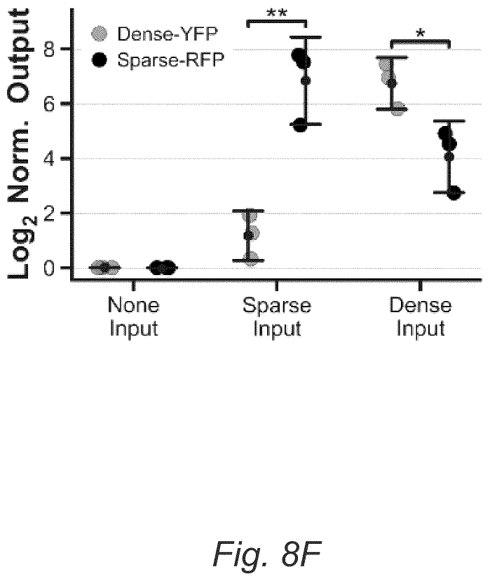

In natural signaling pathways, protein level signaling processes are converted into longer timescale cellular decisions that culminate in phenotypic outcomes³. In a similar manner, the aim was to leverage the FM protein interactions of the system to regulate gene expression and other downstream cellular functions. To adapt the frequency decoder for transcriptional regulation (FIGS. 8A and 8B), efforts were directed toward controlling the recruitment of the VP64 transcriptional activator²⁶ to a DNA-binding domain such as TetR²⁷. In turn, this domain binds to its cognate promoter region (TetO) upstream of a gene of interest allowing VP64 to trigger transcriptional activity. For these experiments, a transcriptional fluorescence reporter construct was employed, consisting of 12×TetO binding sites and the YB-TATA core promoter²⁸ positioned upstream of the gene encoding mScarlet-I²⁹, a red fluorescent protein (RFP). To construct the transcriptional dense decoder (Dense-RFP), the optogenetic components TetR-iLIDfast and sspB-VP64 were used. In parallel, the sparse transcriptional decoder (Sparse-RFP) was implemented using the optogenetic constructs TetR-LOVfast, Zdk-iLIDslow, and sspB-VP64. Consistent with the behavior observed in the ddFP systems, these transcriptional decoder configurations were designed to selectively respond to their corresponding dense or sparse light input patterns, thereby inducing expression of the RFP reporter gene.

As an initial step, an appropriate blue light intensity capable of inducing gene transcription was determined (FIG. 8C). HEK293T cells were transduced with either the TetO-RFP reporter only or the Dense-RFP constructs and exposed to a range of blue light intensities continuously over 24 hours. After exposure, RFP reporter expression levels were quantified from fluorescence microscopy images and normalized to the corresponding negative control group (0 μW/mm²). The reporter only groups exposed to blue light resulted in negligible changes in transcriptional output relative to their negative control group. In contrast, the Dense-RFP groups exposed to blue light intensities at or above 5 μW/mm² exhibited significantly higher reporter output relative to their negative control group. An increasing trend in reporter output was observed with higher blue light intensities. Although a significant increase in expression was noted when the intensity was raised from 10 to 50 μW/mm², a blue light intensity of 10 μW/mm² was selected for subsequent experiments to mitigate potential cellular phototoxicity.

Next, the input pulse frequency response of the transcriptional decoders was examined by varying the pulse period, while maintaining a fixed light intensity of 10 μW/mm² and a pulse width of 1 second (FIG. 8D). HEK293T cells were transduced with either Dense-RFP or Sparse-RFP constructs and exposed to a range of blue light pulse frequencies over 24 hours. Dense-RFP showed increasing gene expression with higher pulse frequencies, reaching optimal induction at 1 Hz (continuous light exposure). On the other hand, the Sparse-RFP groups exhibited a bell-shaped relation, peaking at 0.25 Hz (1s ON/3 s OFF). These results confirmed that the FM behavior of the protein interaction system was transferable towards downstream transcriptional responses, yielding engineered cells that optimally express a gene of interest at specific ranges of pulse frequencies. For subsequent experiments, input frequencies of 0.25 Hz and 1 Hz were selected as the optimal conditions for the sparse and dense decoders, respectively, as these frequencies yielded a significantly high contrast between their corresponding responses.

Although the Sparse-RFP construct exhibited the expected frequency response, it was also confirmed that the signaling function of its LOVfast/Zdk and iLIDslow/sspB components was maintained within the transcriptional regulation setup (FIG. 8E). HEK293T cells were transduced with the TetO-RFP reporter and either TetR-LOVfast+Zdk-VP64 or TetR-iLIDslow+sspB-VP64, and exposed to none, sparse, or dense input over 24 hours. Consistent with the sparse decoder design, the LOVfast regulated system displayed significantly lowered RFP expression for the dense input group relative to the sparse input group. The iLIDslow regulated system also met expectations by triggering high RFP expression during both sparse and dense input relative to the none input group.

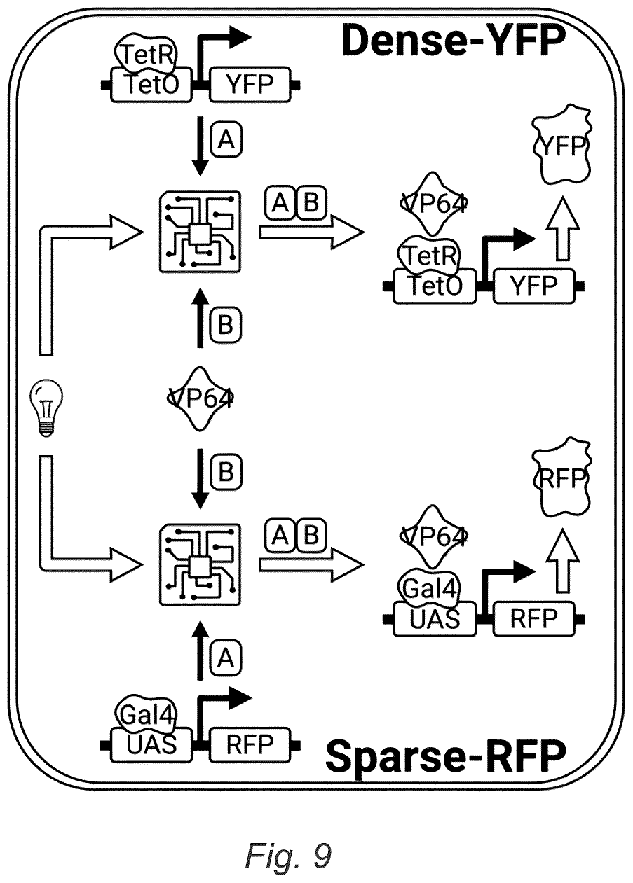

To expand the versatility of the system, a dual frequency-modulated (FM) decoder was developed to enable simultaneous control of two target genes within the same cell (FIG. 9). Given that both decoders shared certain components, the TetR/TetO system in the sparse decoder was replaced with the Gal4/UAS system. Additionally, two distinct fluorescent reporter constructs-TetO-mGold⁽³⁰⁾ and UAS-mScarlet-were employed to generate independent transcriptional readouts for each decoder. Following co-transduction of HEK293T cells with the constructs for both decoders, the transcriptional responses of the combined system were evaluated under conditions of no input, sparse input, and dense input (FIG. 8F). Overall, this combined system displayed frequency selective behavior similar to the previous single decoder results, demonstrating that synthetic multiplexing logic can be engineered within a single cell system, allowing for precise transcriptional control based on light input frequency.

### FM Control of Light Inducible Cell-Cell Immune Responses

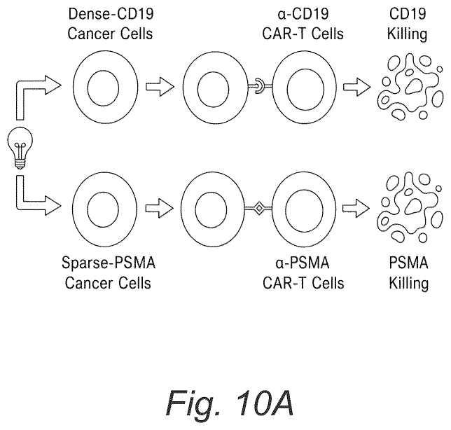

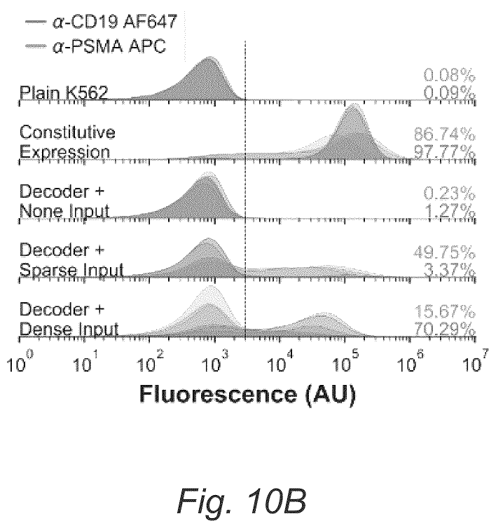

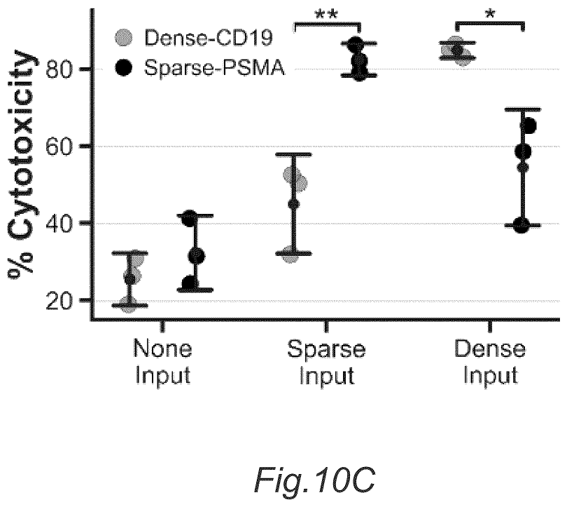

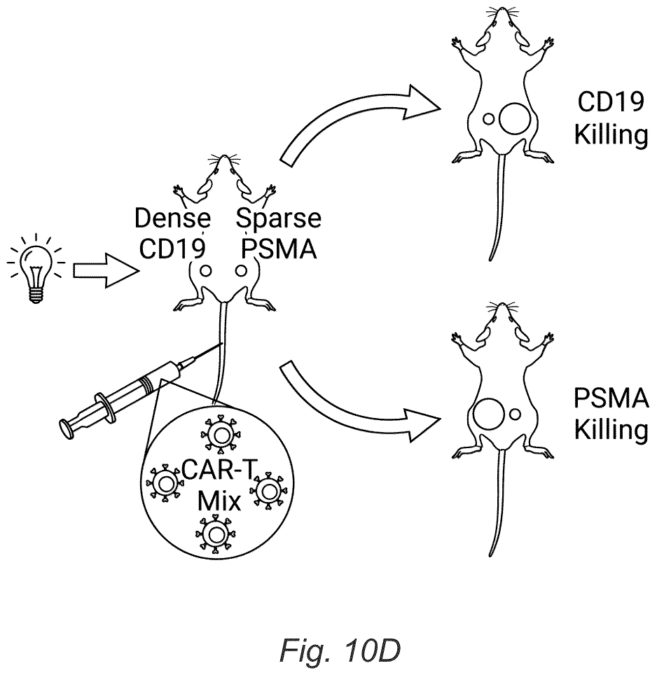

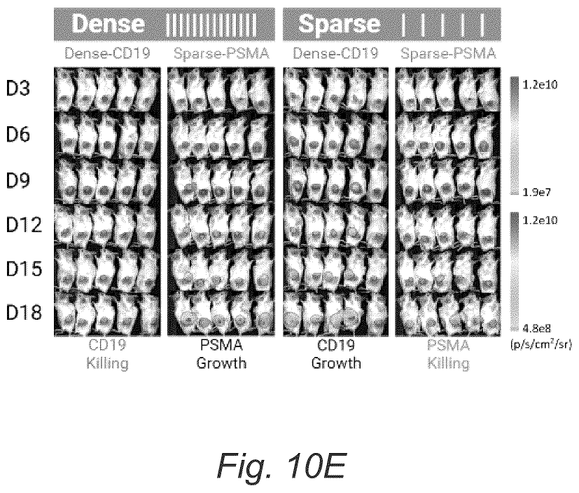

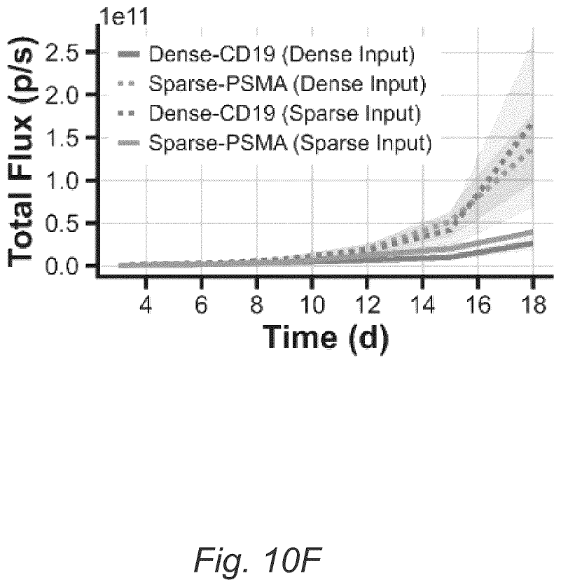

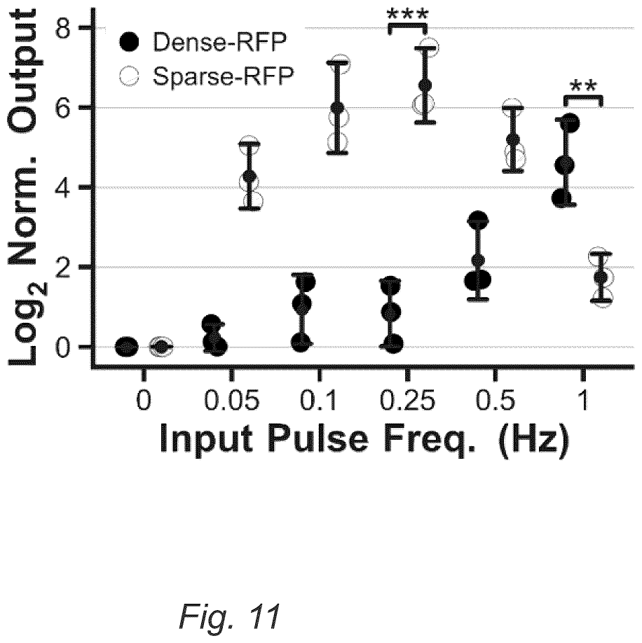

Synthetic development of membrane receptor signaling has been central towards the advancement of cancer immunotherapy^{31,32}. A key focus in this field is regulating the interactions between a diverse range of tumor antigens and chimeric antigen receptors (CARs) to avoid nonspecific immune responses. The applicability of the frequency decoder system was further explored in the context of regulating the expression of multiple antigens to facilitate interactions between tumor and immune cells. The system was adapted to control the expression and membrane presentation of either CD19 or PSMA antigens in K562 tumor cells, depending on the input frequency. Specifically, CD19 expression was linked to dense input stimulation (Dense-CD19), while PSMA expression was driven by sparse input stimulation (Sparse-PSMA) (FIG. 10A). These cells could then be co-cultured with human primary T cells expressing either anti-CD19 or anti-PSMA CAR to assess their ability to trigger antigen specific cytotoxic responses.

The FM transcriptional system was first confirmed to function similarly in K562 cells as it did in HEK293T cells. K562 cells were transduced with either Dense-RFP or Sparse-RFP constructs and exposed to a range of blue light pulse frequencies over 24 hours. This resulted in an RFP response curve that was consistent with the same experiment performed with HEK293T cells.

To implement the FM decoders for regulating antigen presentation, the constructs TetO-CD19 and TetO-PSMA were generated. In addition to the plain K562 negative control group and K562 cells constitutively expressing either CD19 or PSMA antigen as the positive control groups, the Dense-CD19 and Sparse-PSMA transduced groups were exposed to none, sparse, or dense inputs. Subsequently, their antigen production was evaluated using immunostaining and flow cytometry (FIG. 10B). The results showed that the Dense-CD19 cells yielded the highest CD19 antigen expression under dense input while Sparse-PSMA cells had the strongest PSMA expression with sparse input. The induced K562 cells were then co-cultured with the corresponding anti-CD19 or anti-PSMA CAR-T cells, and the cytotoxic responses of the CAR-T cells toward the K562 targets were evaluated (FIG. 10C). A significant difference in cytotoxic efficiency was observed between the two systems at sparse and dense input conditions, consistent with the antigen production results.

Lastly, the system was evaluated in vivo using a bilateral tumor mouse model (FIG. 10D). Mice were injected with Dense-CD19 K562 cells in one flank and Sparse-PSMA K562 cells in the opposite flank. After 8 days, the mice were exposed to either dense or sparse blue light input to induce antigen expression, followed by an intravenous injection of a mixture of both anti-CD19 and anti-PSMA CAR-T cells. Exposure to dense or sparse input triggered substantial antigen presentation and CAR-T killing only in the tumors expressing the corresponding dense or sparse decoders, respectively, resulting in suppressed tumor growth (FIG. 10E, F). Overall, the bilateral tumor model showed the desired frequency selective behavior. On day 18, there was a significant difference in tumor bioluminescence signal between the input matching vs the non-input matching tumors.

### FM Control of Light Inducible Cell-Cell Differentiation Responses

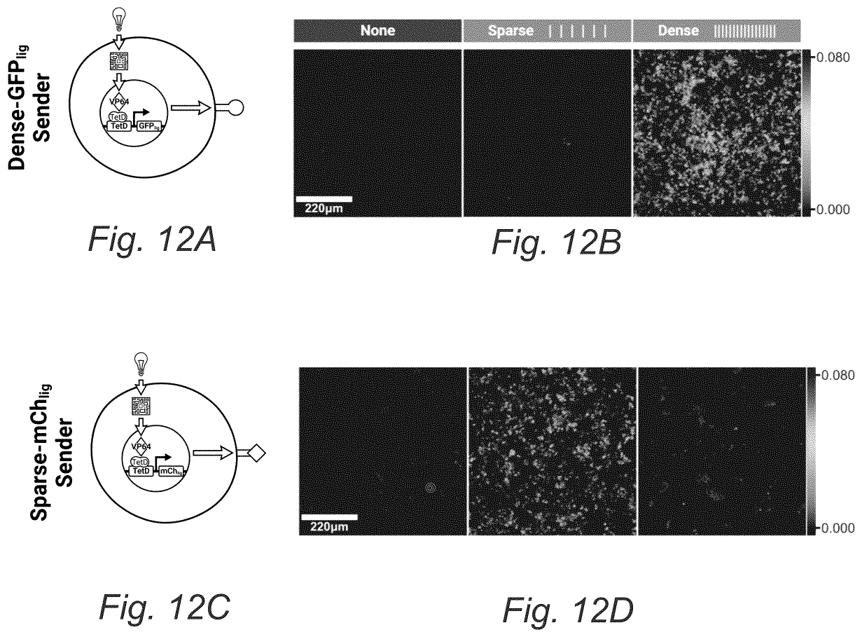

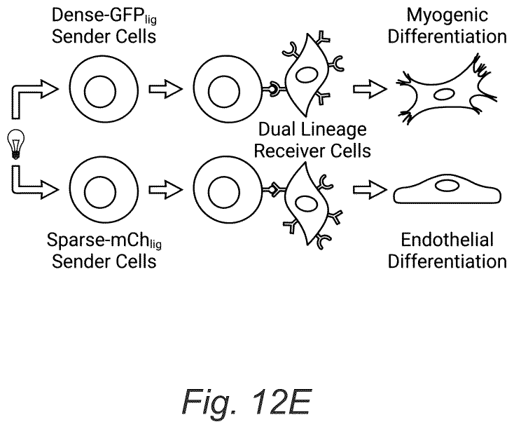

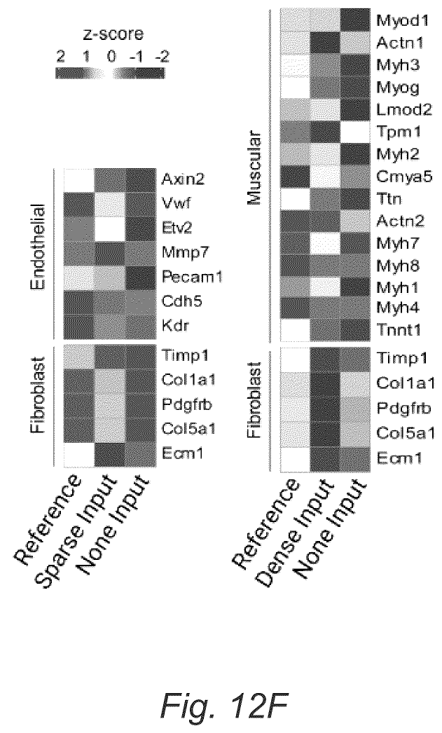

FM control of membrane antigen production can also be applied for triggering other cellular functions such as differentiation. For this application, TetO-GFPligand and TetO-mCherryligand constructs were generated and co-transduced with the corresponding dense or sparse decoder constructs to create Dense-GFPlig and Sparse-mChlig K562 sender cells (FIGS. 12A and 12C). Following a 24-hour exposure of the sender cells to no input, sparse input, or dense input, fluorescence microscopy was conducted to visualize the membrane expression of the GFP and mCherry ligands (FIGS. 12B and 12D). Representative images showed that the FM input response of the sender cells is consistent with the previous results. Dense-GFP_{lig} yielded high GFP_{lig} expression only during dense input while Sparse-mCh_{lig} yielded high mCh_{lig} expression only during sparse input.

Either Dense-GFPlig or Sparse-mChlig sender cells were co-cultured with C3H dual differentiation receiver cells⁽³³⁾, which express both anti-GFPlig and anti-mChlig synNotch receptors that regulate the expression of MyoD and ETV2, respectively (FIG. 12E). Exposing the co-culture to sparse input led to ETV2 expression and promoted differentiation of the C3H fibroblasts into endothelial-like cells. On the other hand, dense input facilitated C3H differentiation into muscular cells through the expression of MyoD. RNA sequencing results from the C3H cells (FIG. 12F) confirmed the overall trends of differentiation, with endothelial marker genes such as Axin2, Vwf, Etv2, Mmp7, and Pecam1, showing increased expression in the group exposed to sparse input. In the dense input treated group, muscular genes such as Myod1, Myog, Myh2, and Myh3, were similarly upregulated. Furthermore, the fibroblast-associated genes Timp1, Col1a1, Pdgfrb, Col5a1, and Ecm1 consistently decreased in the corresponding control groups where no input was applied. The findings were also consistent with the previous public dataset³³ in terms of the differentiation trend, further supporting the effectiveness of the system in directing cellular differentiation.

### DISCUSSION

The experiments set forth above demonstrated an optogenetic platform capable of transducing frequency-modulated (FM) light stimulations into a range of genetic and cellular responses. Through computational modeling and optimization, high-performing and simplified network motifs were identified that satisfied the desired signaling criteria. While previous studies have employed similar computational strategies³⁴⁻³⁸, this work emphasized design practicality. The ProtoSigNet model was intentionally constructed using simple reaction types—reversion, induction, and interaction—that are broadly compatible with diverse cellular signaling components and systems. Owing to its simplicity, the model serves as a strong foundational framework for the integration of additional signaling mechanisms and features, including logic-gated interactions^{39, 40}.

The broader approach of using discrete optimization to evolve a model that can be specified as a set of parameters is another promising area for future development. Alternative modeling methods such as Petri Nets are more suitable for representing hierarchical systems⁴¹⁻⁴³, possibly allowing this optimization approach to be extended to multicellular models. Improved optimization algorithms would also allow larger and more complex models to be manipulated effectively. While NSGA-II has been well-established, a recent benchmark of many state-of-the-art multi-objective optimization algorithms⁴⁴ revealed potential upgrades such as the Nondominated Neighbor Immune Algorithm⁴⁵.

Another key contribution of these experiments was the demonstration that fluorescent biosensors can be effectively utilized to streamline the implementation of signaling circuits in cells by aiding in component selection and validating signaling functionality. The ddFP biosensor system was employed to characterize the signaling dynamics of optogenetic protein variants and to validate the frequency-modulated (FM) decoders. Notably, the RA and B3 domains provided clear and robust dynamic fluorescence readouts without the need to optimize parameters such as the protein linker. Given that protein-protein interactions represent a common signaling modality, ddFP and similar biosensor systems⁴⁶ offer broad applicability in the development of synthetic biology platforms.

Lastly, the protein interaction outputs of the system were successfully translated into control over gene transcription and multicellular responses. Gene transcription represents a particularly attractive target, as the gene of interest can be readily substituted to accommodate a wide range of biological applications. For instance, cell fate decisions could be directed through the expression of specific differentiation factors⁴⁷, and receptor-mediated responses in other cells could be triggered by expressing designated ligands or antigens. Aside from that, a key strength of the system is the ability to control fast timescale interactions between any two desired cellular elements, in principle, which sets it apart from previous transcription-based systems^{35,48}. This underscores the potential application of the FM decoder system for controlling responses based on the reconstitution of engineered split proteins⁴⁹ or the recruitment of protein effectors to specific subcellular compartments such as GTPases to the cell membrane¹⁷.

### Experimental Model and Subject Details

### General Cell Culture

HEK293T cells (ATCC CRL-3216) and C3H/10T1/2 Clone 8 cells (ATCC CCL-226) were cultured in Dulbecco's Modified Eagle Medium (DMEM) (Gibco 11995065) supplemented with 10% (v/v) fetal bovine serum (FBS) (Thermo Fisher Scientific 10438026), 100 units/mL penicillin, and 100 μg/mL streptomycin (Gibco 15140122). K562 cells (ATCC) were cultured in Roswell Park Memorial Institute Medium (RPMI 1640) (Gibco 22400105) supplemented with 10% FBS, 100 units/mL penicillin, and 100 μg/mL streptomycin. Primary human T cells were cultured in RPMI 1640 supplemented with 100 U/mL recombinant human IL-2 (PeproTech 200-02). All cells were kept in a humidified incubator maintained at 37 °C and 5% CO₂.

### Isolation, Culture, and Transduction of Primary Human T Cells

Human peripheral blood mononuclear cells (PBMCs) were isolated from buffy coats (San Diego Blood Bank) using Lymphocyte Separation Medium (Corning 25-072-CV) following the manufacturer's instructions and CD3⁺ primary human T cells were isolated using Pan T Cell Isolation Kit (Miltenyi Biotec 130-096-535). For transduction, T cells were activated with Dynabeads Human T-Expander CD3/CD28 (Gibco 11141D) for 72 hours before a lentivirus cocktail concentrated using PEG-it (SBI LV825A-1) was added at a multiplicity of infection of 10 followed by spinoculation in a 24-well plate coated with Retronectin (Takara Bio T100B) at 1800 g, 32 °C for 1 hour.

### In Vivo Bilateral Tumor Model

NSG mice were subcutaneously injected with 1×10⁶ dense-CD19 or sparse-PSMA K562 cells on the left and right flank, respectively. On day 8 after tumor inoculation, mice were treated with dense or sparse blue light input for 12 hours as indicated. On day 9, a total of 2×10⁶ anti-CD19 CAR T cells and 2×10⁶ anti-PSMA CAR T cells per mouse were injected via tail vein. Mice were treated with another dose of dense or sparse blue light input on day 13. Tumor growth was monitored by bioluminescence imaging (BLI) with Ami HTX imaging system (Spectral Instruments Imaging).

### Method Details

### Plasmid Construction

Linear DNA fragments were generated via PCR using Q5 DNA polymerase (NEB M0491L). After PCR, the correct DNA fragments were isolated using agarose gel electrophoresis and a DNA gel recovery kit (Zymo D4007). Synthetic gene constructs were assembled into a pHR lentiviral plasmid vector (Addgene #79129) using Gibson Assembly (NEB E2611L) and DpnI (NEB R0176S) digestion was performed to eliminate template DNA. NEB5α competent cells (NEB C2987I) were transformed with the assembly product and plasmid DNA was isolated using a miniprep kit (Sigma PLN350) for downstream mammalian cell experiments. All constructs were verified using Sanger sequencing (Genewiz) and sufficient plasmid concentration and purity (260/280>1.8, 260/230>2.0) were confirmed using a Nanodrop 2000c Spectrophotometer (ThermoFisher ND2000C).

### Lentivirus Production and Transduction

For lentivirus production, an appropriate number of HEK293T cells were seeded in 6 well plates (Corning 3506) to yield 70%-90% confluency the following day. Co-transfection was performed using Lipofectamine 3000 (Thermo Fisher Scientific L3000015) with 1 μg transfer vector (pHR backbone derived from Addgene #79129), 0.75 μg psPAX2 (Addgene #12260), and 0.25 μg pMD2.G (Addgene #12259) prepared in Opti-MEM (Gibco 31985070) according to the manufacturer's instructions. Lentivirus containing supernatant was collected 48 hours post-transfection, filtered through a 0.45 μm filter (Millipore SLHV033RS), and stored at −80 °C. For transduction, approximately 5×10⁵ target cells were seeded in 6 well plates. The next day, 250 μL virus supernatant of each construct was added to the cells. Cell culture media was replaced the following day.

### Fluorescence Microscopy Setup

Fluorescence images were acquired using a Photometrics Prime 95B sCMOS camera on a Nikon Eclipse Ti inverted microscope. Samples were maintained at 37 °C and 5% CO₂ on a Prior H117 stage with a custom stage incubator. Timelapse imaging and blue light stimulation were performed using a custom Python automation script and MicroManager⁵⁰.

Imaging of ddFP Biosensors

48 hours after transduction, approximately 3×10⁵ HEK293T cells were seeded onto a 29 mm dish containing a 20 mm #1.5 glass insert (Cellvis D29-20-1.5-N) coated with 10 μg/mL fibronectin (Sigma F1141) and kept in an incubator at 37 °C and 5% CO₂ for 2-4 hours to adhere to the insert before imaging. Blue light input was administered to the cells with a [470/30 nm EX, 595 nm LP, 650/100 nm EM] filter set through a Plan Apo 100×1.45 NA Oil objective. The light intensity at the objective was measured using a handheld light power meter (Newport 843-R) and an intensity of 20 μW/mm² was used for all ddFP experiments unless denoted otherwise. The same objective and a [580/20 nm EX, 595 nm LP, 650/100 nm EM] filter set were used to image the ddFP fluorescence.

### Imaging of FP Expression Reporters

48 hours after transduction, approximately 2×10⁵ HEK293T were plated per well of a black, glass bottom 24-well plate (Labnet International 33-624X). The LAVA 24-well optogenetic illumination device⁵¹ was used to independently expose each well to pre-programmed patterns of blue light for 24 hours. Unless denoted otherwise, a blue light intensity of 10 μW/mm² was used. After the 24 hours, snapshot images were collected using a Plan 10×0.25 NA Dry objective with the filter sets [515/10 nm EX, 525 nm LP, 545/30 nm EM] for mGold and [580/20 nm EX, 595 nm LP, 650/100 nm EM] for mScarlet-I.

Imaging of GFP_{lig} and mCh_{lig}

48 hours after transduction, approximately 2×10⁵ K562 were plated per well of a black, glass bottom 24-well plate (Labnet International 33-624X). The LAVA 24-well optogenetic illumination device⁵¹ was used to independently expose each well to pre-programmed patterns of blue light for 24 hours. Unless denoted otherwise, a blue light intensity of 10 μW/mm² was used. After the 24 hours, images were collected using a Plan 10×0.25 NA Dry objective with the filter sets [480/30 nm EX, 505 nm LP, 535/40 nm EM] for GFP_{lig} and [580/20 nm EX, 595 nm LP, 650/100 nm EM] for mCh_{lig}.

### Staining and Flow Cytometry

Staining of cell surface markers (hCD19 and hPSMA) for flow cytometry was performed using fluorophore-conjugated antibodies according to the manufacturer's protocol. Cells were washed and resuspended in 100 L PBS containing the suggested amounts of antibodies, incubated in the dark at room temperature for 15 minutes, and washed three times (1 mL PBS, centrifuged at 350 g for 5 minutes) before flow cytometry (BD Accuri C6) was performed. Gating was based on non-transduced K562 cells with the same staining. For GFP_{lig} and mCh_{lig}, cells were resuspended in PBS and flow cytometry (BD Accuri C6) was performed. Flow cytometry data was analyzed using cytoflow⁵².

### Luciferase-Based Cytotoxicity Assay

K562 cells transduced with dense-CD19 or sparse-PSMA decoder constructs were exposed to the specified blue light inputs for 24 hours in black, glass-bottom 24-well plates: None Input, Sparse Input (0.25 Hz or is ON/3 s OFF), and Dense Input (1 Hz or continuously ON). The None Input group was used as a “K562 only” negative control. For the other groups, a constant number of 5×10⁴ Fluc+ K562 cells were mixed with engineered primary human T cells (pre-washed and resuspended with complete RPMI without IL-2) at an effector-to-target (E:T) ratio of 3:1. The mixtures were then cultured in round bottom 96-well plates for 24 hours, centrifuged to remove the supernatant and assayed with the Bright-Glo Luciferase Assay System (Promega E2610) following the manufacturer's instructions to quantify the luminescence of each sample. The cytotoxicity (%) of sample X was calculated as (1−Luminescence of X/Luminescence of “K562 only”)×100.

### Dual Differentiation Co-Culture and Light Stimulation

On day 0, 5×10⁴ C3H dual differentiation receiver cells were plated per well of a black, glass bottom 24-well plate (Labnet International 33-624X). On day 1, 1×10⁵ Dense-GFP_{lig} or Sparse-mCh_{lig} K562 sender cells were added per well to the corresponding groups (each group pooling 6 wells). The LAVA 24-well optogenetic illumination device⁵¹ was used to independently expose each well to pre-programmed patterns of blue light for 24 hours. Unless denoted otherwise, a blue light intensity of 10 μW/mm² was used. On day 2, light stimulation was halted for 24 hours. On day 3, the cells were re-stimulated with the same input pattern for another 24 hours. Immediately after, C3H dual differentiation receiver cells were harvested and assayed for differentiation response.

RNA Extraction and cDNA Synthesis

C3H dual differentiation receiver cells were harvested from co-culture experiments and Total RNA was extracted using the Zymo Direct-Zol™ RNA Microprep Kit (Zymo Research, R2062), following the manufacturer's protocol. RNA integrity and concentration were assessed using a NanoDrop spectrophotometer (Thermo Fisher Scientific), ensuring A260/A280 ratios between 1.8 and 2.0. On-column DNase I treatment was performed to remove genomic DNA contamination. Complementary DNA (cDNA) was synthesized from 1 g of total RNA using the SuperScript™ IV Reverse Transcriptase Kit (Thermo Fisher Scientific, 18090050). The reaction was performed according to the manufacturer's instructions, using random hexamer primers in a L reaction volume. Reverse transcription was carried out at 50 °C for 10 minutes, followed by enzyme inactivation at 80 °C for 10 minutes.

### RT-qPCR

RT-qPCR was performed using the Bio-Rad CFX96 Real-Time PCR System with SsoAdvanced Universal SYBR Green Supermix (Bio-Rad) in 10 L reactions containing 500 nM of each primer and 2 L of cDNA template. Relative gene expression levels were determined using the ΔΔCt method, normalizing target gene expression to the housekeeping gene 18S ribosomal RNA.

### RNA Sequencing Data Analysis

C3H dual differentiation receiver cells were sorted based on BFP/miRFP fluorescence after co-culturing with either K562 Dense-GFP_{lig} or Sparse-mCh_{lig} sender cells and exposing to dense or sparse blue light stimulation, respectively. Total RNA was collected using Zymo Direct-Zol™ RNA Microprep Kit (Zymo Research, R2062) and subjected to mRNA Sequencing (Novogene). Quality control was performed using fastp software to remove adapters and low-quality reads. Hisat2 version 2.0.5 was used for mapping reads to mouse genome (mm39) and transcripts quantification was performed using featureCounts 1.5.0-p3. Public RNA Sequencing data (GSE269404) was processed following the same pipeline. Batch correction of public data set and in-house generated data set was performed using sva version 3.54.0 and normalized using DESeq2 version 1.20.0 in the R programming language. Heatmaps were generated using normalized counts with pheatmap version 1.0.12. Differential expression gene (DEG) analysis was performed using DEGseq Release (3.20) in the R programming language with an adjusted p value of <0.05. DEGs were subjected to gene ontology analysis using DAVID webserver and GSEA analysis through the GSEA desktop application.

### Mathematical Modeling

### General Signaling Model

The Prototyping Signaling Network model is defined by the following equation:

$$\frac{{dX}_{i}}{dt} = {kr}_{i} \cdot {Xr}_{i} + {ku}_{i} \cdot {Xu}_{i} \cdot {u}_{i} + {\sum }_{j = 1}^{n} {kX}_{ij} \cdot {Xy}_{j} \cdot {X}_{i}$$

The model state variables (Xᵢ) represent cell components (e.g., optogenetic proteins) and vary between the range [0, 1] where 0 can be considered some inactive state (e.g., closed conformation) and 1 can be considered some active state (e.g., open conformation).

The variable uᵢ represents the intensity of stimuli input signals acting on each component Xᵢ over time. It can be either a 2D array (N×t) specifying separate stimuli inputs affecting each node/component (N) or a 1D array (1×t) specifying a single stimulus input affecting all nodes/components.

Model parameters specify the reactions and network structure of the system. For a system of N nodes, there are a total of N reversion+N induction+N² interaction parameters.

Reversion rates (krᵢ∈(−∞, ∞); krᵢ≠0): an intrinsic reaction that drives Xᵢ towards either inactive (0) or active (1) state.

Stimuli induction rates (kuᵢ∈[0, ∞)): a reaction from an external stimulus that drives Xᵢ in the opposite direction of its reversion reaction.

$${ku}_{i} = \{ \begin{matrix}- {ku}_{i} & if {ku}_{i} \cdot {kr}_{i} < 0 \\ {ku}_{i} & if {ku}_{i} \cdot {kr}_{i} \geq 0\end{matrix}$$

Component interaction rates (kXᵢⱼ∈(−∞, ∞)): reactions towards a component (i) from another component (j).

Initial conditions are determined by krᵢ:

$${X}_{0 i} = \{ \begin{matrix}0 & if {kr}_{i} < 0 \\ 1 & if {kr}_{i} \geq 0\end{matrix}$$

The direction/polarity of reversion, induction, and interaction reactions depend on their sign: Negative=repression reaction driving Xᵢ towards 0
Positive=activation reaction driving Xᵢ towards 1

The strength of a reaction depends on its absolute value. Therefore, a parameter value of 0 equates to simply the absence of that particular reaction.

Each reaction affecting a given node (Xᵢ) also accounts for how far it is from the inactive or active state that the reaction is driving it towards.

$${Xr}_{i} = \{ \begin{matrix}{X}_{i} & {kr}_{i} < 0 \\ 1 - {X}_{i} & {kr}_{i} \geq 0\end{matrix}$$

$${Xu}_{i} = \{ \begin{matrix}{X}_{i} & {ku}_{i} < 0 \\ 1 - {X}_{i} & {ku}_{i} \geq 0\end{matrix}$$

$${Xy}_{i} = \{ \begin{matrix}{X}_{i} & {kX}_{ij} < 0 \\ 1 - {X}_{i} & {kX}_{ij} \geq 0\end{matrix}$$

### Optogenetic Signaling Model

The Optogenetic Protein Interaction (OptoPI) model includes individual models for light inducible dissociation (LOV/Zdk) and light inducible dimerization (iLID/sspB).

### LOV AND ILID TERMS

Aᵢ=inactive/closed conformation LOV or iLID
Aₐ=active/open conformation LOV/iLID
B=Zdk or sspB
AB=LOV-Zdk or iLID-sspB bound complex
u(t)=blue light input signal (0 or 1)
v(t)={0 if u=0 else 1}
kₗ=blue light induction rate
k_{d}=dark reversion rate
k_{b}=binding rate

### LOV/Zdk Equations:

$$\frac{{dA}_{i}}{dt} = - u \cdot {k}_{l} \cdot {A}_{i} + v \cdot {k}_{d} \cdot {A}_{a} - {k}_{b} \cdot {A}_{i} \cdot B$$

$$\frac{{dA}_{a}}{dt} = u \cdot {k}_{l} \cdot {A}_{i} - v \cdot {k}_{d} \cdot {A}_{a} + u \cdot {k}_{l} \cdot AB$$

$$\frac{dB}{dt} = - {k}_{b} \cdot {A}_{i} \cdot B + u \cdot {k}_{l} \cdot AB$$

$$\frac{dAB}{dt} = {k}_{b} \cdot {A}_{i} \cdot B - u \cdot {k}_{l} \cdot AB$$

iLID/sspB Equations:

$$\frac{{dA}_{i}}{dt} = - u \cdot {k}_{l} \cdot {A}_{i} + v \cdot {k}_{d} \cdot {A}_{a} + v \cdot {k}_{d} \cdot AB$$

$$\frac{{dA}_{a}}{dt} = u \cdot {k}_{l} \cdot {A}_{i} - v \cdot {k}_{d} \cdot {A}_{a} - {k}_{b} \cdot {A}_{a} \cdot B$$

$$\frac{dB}{dt} = - {k}_{b} \cdot {A}_{a} \cdot B + v \cdot {k}_{d} \cdot AB$$

$$\frac{dAB}{dt} = {k}_{b} \cdot {A}_{a} \cdot B - v \cdot {k}_{d} \cdot AB$$

The reactions and parameters from these models were leveraged to compose a sparse decoder model.

### Sparse Decoder Terms:

A=LOVfast
B=Zdk-iLIDslow
C=sspB
subscript i=inactive/closed conformation
subscript a=active/open conformation
XY=component X in complex with component Y
X0=initial concentration of X
u(t)=blue light input signal (0 or 1)
v(t)={0 if u=0 else 1}
kₗ₁=LOVfast blue light induction rate
k_{d1}=LOVfast dark reversion rate
k_{b1}=LOVfast binding rate
kₗ₂=iLIDslow blue light induction rate
k_{d2}=iLIDslow dark reversion rate
k_{b2}=iLIDslow binding rate

### Sparse Decoder Equations:

$$\frac{{dA}_{i}}{dt} = - u \cdot {k}_{l 1} \cdot {A}_{i} + v \cdot {k}_{d 1} \cdot {A}_{a} - {k}_{b 1} \cdot {A}_{i} \cdot {B}_{i} - {k}_{b 1} \cdot {A}_{i} \cdot {B}_{a} - {k}_{b 1} \cdot {A}_{i} \cdot {B}_{a} C$$

$$\frac{{dA}_{a}}{dt} = u \cdot {k}_{l 1} \cdot {A}_{i} - v \cdot {k}_{d 1} \cdot {A}_{a} + u \cdot {k}_{l 1} \cdot {A}_{i} {B}_{i} + u \cdot {k}_{l 1} \cdot {A}_{i} {B}_{a} + u \cdot {k}_{l 1} \cdot {A}_{i} {B}_{a} C$$

$$\frac{{dB}_{i}}{dt} = - u \cdot {k}_{l 2} \cdot {B}_{i} + v \cdot {k}_{d 2} \cdot {B}_{a} - {k}_{b 1} \cdot {A}_{i} \cdot {B}_{i} + v \cdot {k}_{d 2} \cdot {B}_{a} C$$

$$\frac{{dB}_{a}}{dt} = u \cdot {k}_{l 2} \cdot {B}_{i} - v \cdot {k}_{d 2} \cdot {B}_{a} - {k}_{b 1} \cdot {A}_{i} \cdot {B}_{a} - {k}_{b 2} \cdot {B}_{a} \cdot C + u \cdot {k}_{l 1} \cdot {A}_{i} {B}_{i} + u \cdot {k}_{l 1} \cdot {A}_{i} {B}_{a}$$

$$\frac{dC}{dt} = - {k}_{b 2} \cdot {B}_{a} \cdot C - {k}_{b 2} \cdot {A}_{i} {B}_{a} \cdot C + v \cdot {k}_{d 2} \cdot {B}_{a} C + v \cdot {k}_{d 2} \cdot ( {A}_{i} {B}_{a} C - {A}_{i} {B}_{a} C 0 )$$

$$\frac{{dA}_{i} {B}_{i}}{dt} = {k}_{b 1} \cdot {A}_{i} \cdot {B}_{i} - u \cdot {k}_{l 1} \cdot {A}_{i} {B}_{i} + v \cdot {k}_{d 2} \cdot ( {A}_{i} {B}_{a} C - {A}_{i} {B}_{a} C 0 )$$

$$\frac{{dA}_{i} {B}_{a}}{dt} = {k}_{b 1} \cdot {A}_{i} \cdot {B}_{a} - {k}_{b 2} \cdot {A}_{i} {B}_{a} \cdot C - u \cdot {k}_{l 1} \cdot {A}_{i} {B}_{a}$$

$$\frac{{dB}_{a} C}{dt} = {k}_{b 2} \cdot {B}_{a} \cdot C - {k}_{b 1} \cdot {A}_{i} \cdot {B}_{a} C - v \cdot {k}_{d 2} \cdot {B}_{a} C + u \cdot {k}_{l 1} \cdot {A}_{i} {B}_{a} C$$

$$\frac{{dA}_{i} {B}_{a} C}{dt} = {k}_{b 2} \cdot {A}_{i} {B}_{a} \cdot C + {k}_{b 1} \cdot {A}_{i} \cdot {B}_{a} C - v \cdot {k}_{d 2} \cdot ( {A}_{i} {B}_{a} C - {A}_{i} {B}_{a} C 0 ) - u \cdot {k}_{l 1} \cdot {A}_{i} {B}_{a} C$$

### ODE Model Simulation

Dynamic simulations were generated using the Python SciPy library⁵³. Simulations were performed by numerically integrating the equations using the BDF solver with relative tolerance of 1e-5, absolute tolerance of 1e-5, and max step size of 0.1.

### ODE Model Parameter Estimation

To estimate parameter values for the OptoPI models, the residual between the model response ([AB] bound complex) and the corresponding ddFP data was minimized using the differential evolution algorithm from LMFIT⁵⁴.

residual=(y_{model}−y_{mean,data})/y_{sem,data}

### FM Decoder Design Optimization

Optimization of the general signaling model for the frequency decoder design problem was performed using the Non-dominated Sorting Genetic Algorithm 2 (NSGA-II) implemented based on the original publication¹⁹. The algorithm evolves a population of model candidates/individuals represented as arrays of parameters. To derive an FM decoder motif, an initial 5-node model containing 35 parameters was specified:
krᵢ, ku₁, kX₁₁, kX₁₂, kX₁₃, kX₁₄, kX₁₅
kr₂, ku₂, kX₂₁, kX₂₂, kX₂₃, kX₂₄, kX₂₅
kr₃, ku₃, kX₃₁, kX₃₂, kX₃₃, kX₃₄, kX₃₅
kr₄, ku₄, kX₄₁, kX₄₂, kX₄₃, kX₄₄, kX₄₅
kr₅, ku₅, kX₅₁, kX₅₂, kX₅₃, kX₅₄, kX₅₅

For each model candidate, the values for each parameter are randomly drawn from the following parameter spaces:

krᵢ∈[−10,−1,−0.1,−0.01,0.01,0.1,1,10]

kuᵢ∈[0,0.01,0.1,1,10]

kXᵢⱼ∈[−10,−1,−0.1,−0.01,0,0.01,0.1,1,10]

For each NSGA-II optimization run, an initial population of M model candidates was generated by selecting parameters using the quasi-random Halton sampling method⁵⁵. In each generation, the dynamic response of every model candidate was simulated over a fixed timespan (t=0 to t=120, with a time step of dt=1) using its respective parameter set and the defined input profile:
None: u=0 from t=0 to t=40
Sparse: repeating cycles of u=1 for 1 time unit, u=0 for 9 time units from t=40 to t=80
Dense: u=1 from t=80 to t=120

For the optimization objectives, both the simplicity of the gene circuit motif (obj1) and the performance of its FM decoder signaling function (obj2) was maximized:

obj1=count of parameters equal to zero

obj2=X̅₁̅(t_{dense})−X̅₁̅(tₛₚₐᵣₛₑ)−X̅₁̅(tₙₒₙₑ)+X₂(tₛₚₐᵣₛₑ)−X₂(t_{dense})−X₂(tₙₒₙₑ)

where X₁ and X₂ are defined as the output variables for the dense and sparse decoders, respectively. Objective 1 ranges between 0 and 30 since there are 35 parameters in a 5-component model but the 5 reversion parameters are always nonzero. Objective 2 ranges between −4 and 2 since each Xᵢ varies between 0 and 1. Both objective scores were rescaled between 0 and 1 based on their ranges.

### Data and Statistical Analysis

### Image Analysis

Fluorescence images were processed using the Python Scikit-Image library⁵⁶. The background fluorescence level was subtracted by applying a gaussian filter over the image and subtracting this from the raw image. For quantification of ddFP biosensor timelapse data, the fluorescence intensity at a timepoint was calculated by taking the mean across all pixel values in the image frame (1200×1200 px). For quantification of light-inducible gene expression snapshot data, the fluorescence intensity of each well/group was calculated as the mean across all pixel values in an image frame (1200×1200 px) and averaged across all snapshots taken for that sample.

### Calculation of Mean Output Vs Input Pulse Frequency

The corresponding model was simulated for a fixed span of time (t=0 to t=120) with 1 time unit intervals using an FM input pattern defined in terms of [start: stop: interval) as [period: 121: period) where period=1/frequency and pulse width=1 time unit. Thus, a period of 1 yields a 1 Hz input pattern i.e., 1 time unit pulse width per 1 time unit period which equates to continuous input. Since the input is defined as [period: 121: period], the longest input period tested (121) would start outside the time frame which is equivalent to applying no input.

### Data Visualization and Statistical Testing

Plots and data visualizations were generated using the Python packages Numpy⁵⁷, Pandas⁵⁸, Scipy⁵³, and Matplotlib⁵⁹. A two-tailed Student's t-test was calculated using the Scipy stats.ttest_ind function to test the differences between experimental groups for statistical significance. Schematic illustrations and figures were created with BioRender.com.

While exemplary embodiments are described above, it is not intended that these embodiments describe all possible forms of the disclosure. Rather, the words used in the specification are words of description rather than limitation, and it is understood that various changes may be made without departing from the spirit and scope of the disclosure. Additionally, the features of various implementing embodiments may be combined to form further embodiments of the disclosure.

### REFERENCES

1. Shakiba, N., Jones, R. D., Weiss, R. & Del Vecchio, D. Context-aware synthetic biology by controller design: Engineering the mammalian cell. Cell Syst 12, 561-592 (2021).
2. Lim, W. A. Designing customized cell signalling circuits. Nat. Rev. Mol. Cell Biol. 11, 393-403 (2010).
3. Purvis, J. E. & Lahav, G. Encoding and decoding cellular information through signaling dynamics. Cell 152, 945-956 (2013).
4. Li, P. & Elowitz, M. B. Communication codes in developmental signaling pathways. Development 146, dev170977 (2019).
5. Utzny, C., Faroudi, M. & Valitutti, S. Frequency encoding of T-cell receptor engagement dynamics in calcium time series. Biophys. J. 88, 1-14 (2005).
6. O'Donoghue, G. P. et al. T cells selectively filter oscillatory signals on the minutes timescale. Proc. Natl. Acad. Sci. U.S.A. 118, (2021).
7. Harton, M. D., Koh, W. S., Bunker, A. D., Singh, A. & Batchelor, E. p 53 pulse modulation differentially regulates target gene promoters to regulate cell fate decisions. Mol. Syst. Biol. 15, e8685 (2019).
8. Albeck, J. G., Mills, G. B. & Brugge, J. S. Frequency-modulated pulses of ERK activity transmit quantitative proliferation signals. Mol. Cell 49, 249-261 (2013).
9. Bettenworth, V. et al. Frequency modulation of a bacterial quorum sensing response. Nat. Commun. 13, 2772 (2022).
10. Wang, S.-W. & Tang, L.-H. Emergence of collective oscillations in adaptive cells. Nat. Commun. 10, 5613 (2019).
11. Levine, J. H., Lin, Y. & Elowitz, M. B. Functional roles of pulsing in genetic circuits. Science 342, 1193-1200 (2013).
12. Minas, G. et al. Multiplexing information flow through dynamic signalling systems. PLoS Comput. Biol. 16, e1008076 (2020).
13. Ryu, H. et al. Frequency modulation of ERK activation dynamics rewires cell fate. Mol. Syst. Biol. 11, 838 (2015).
14. Ender, P. et al. Spatiotemporal control of ERK pulse frequency coordinates fate decisions during mammary acinar morphogenesis. Dev. Cell 57, 2153-2167.e6 (2022).
15. Spiltoir, J. I. & Tucker, C. L. Photodimerization systems for regulating protein-protein interactions with light. Curr. Opin. Struct. Biol. 57, 1-8 (2019).
16. Redchuk, T. A. et al. Optogenetic regulation of endogenous proteins. Nat. Commun. 11, 605 (2020).
17. Inaba, H., Miao, Q. & Nakata, T. Optogenetic control of small GTPases reveals RhoA mediates intracellular calcium signaling. J. Biol. Chem. 100290 (2021).
18. He, L. et al. Circularly permuted LOV2 as a modular photoswitch for optogenetic engineering. Nat. Chem. Biol. (2021) doi:10.1038/s41589-021-00792-9.
19. Deb, K., Pratap, A., Agarwal, S. & Meyarivan, T. A fast and elitist multiobjective genetic algorithm: NSGA-II. IEEE Trans. Evol. Comput. 6, 182-197 (2002).
20. Ngatchou, P., Zarei, A. & El-Sharkawi, A. Pareto Multi Objective Optimization, in Proceedings of the 13th International Conference on, Intelligent Systems Application to Power Systems 84-91 (IEEE, 2005).
21. Guntas, G. et al. Engineering an improved light-induced dimer (iLID) for controlling the localization and activity of signaling proteins. Proc. Natl. Acad. Sci. U.S.A. 112, 112-117 (2015).
22. Wang, H. et al. LOVTRAP: an optogenetic system for photoinduced protein dissociation. Nat. Methods 13, 755-758 (2016).
23. Zayner, J. P. & Sosnick, T. R. Factors that control the chemistry of the LOV domain photocycle. PLoS One 9, e87074 (2014).
24. Zimmerman, S. P., Kuhlman, B. & Yumerefendi, H. Chapter Eight—Engineering and Application of LOV2-Based Photoswitches. in Methods in Enzymology (ed. Pecoraro, V. L.) vol. 580 169-190 (Academic Press, 2016).
25. Alford, S. C., Abdelfattah, A. S., Ding, Y. & Campbell, R. E. A fluorogenic red fluorescent protein heterodimer. Chem. Biol. 19, 353-360 (2012).
26. Beerli, R. R., Segal, D. J., Dreier, B. & Barbas, C. F., 3rd. Toward controlling gene expression at will: specific regulation of the erbB-2/HER-2 promoter by using polydactyl zinc finger proteins constructed from modular building blocks. Proc. Natl. Acad. Sci. U.S.A. 95, 14628-14633 (1998).
27. Gossen, M. & Bujard, H. Tight control of gene expression in mammalian cells by tetracycline-responsive promoters. Proc. Natl. Acad. Sci. U.S.A. 89, 5547-5551 (1992).
28. Ede, C., Chen, X., Lin, M.-Y. & Chen, Y. Y. Quantitative Analyses of Core Promoters Enable Precise Engineering of Regulated Gene Expression in Mammalian Cells. ACS Synth. Biol. 5, 395-404 (2016).
29. Bindels, D. S. et al. mScarlet: a bright monomeric red fluorescent protein for cellular imaging. Nat. Methods 14, 53-56 (2017).
30. Lee, J. et al. Versatile phenotype-activated cell sorting. Sci. Adv. 6, eabb7438 (2020).
31. Lim, W. A. & June, C. H. The Principles of Engineering Immune Cells to Treat Cancer. Cell 168, 724-740 (2017).
32. Roybal, K. T. et al. Engineering T Cells with Customized Therapeutic Response Programs Using Synthetic Notch Receptors. Cell 167, 419-432.e16 (2016).
33. Garibyan, M. et al. Engineering programmable material-to-cell pathways via synthetic notch receptors to spatially control differentiation in multicellular constructs. Nat. Commun. 15, 5891 (2024).
34. Ravindran, P. T., McFann, S., Thornton, R. H. & Toettcher, J. E. A synthetic gene circuit for imaging-free detection of signaling pulses. Cell Syst 13, 131-142.e13 (2022).
35. Lormeau, C., Rudolf, F. & Stelling, J. A rationally engineered decoder of transient intracellular signals. Nat. Commun. 12, 1886 (2021).
36. Gerardin, J., Reddy, N. R. & Lim, W. A. The Design Principles of Biochemical Timers: Circuits that Discriminate between Transient and Sustained Stimulation. Cell Syst (2019) doi:10.1016/j.cels.2019.07.008.
37. Smith, R. W., van Sluijs, B. & Fleck, C. Designing synthetic networks in silico: a generalised evolutionary algorithm approach. BMC Syst. Biol. 11, 118 (2017).
38. Nielsen, A. A. K. et al. Genetic circuit design automation. Science 352, aac7341 (2016).
39. Gao, Y., Wang, L. & Wang, B. Customizing cellular signal processing by synthetic multi-level regulatory circuits. Nat. Commun. 14, 8415 (2023).
40. Xie, M. & Fussenegger, M. Designing cell function: assembly of synthetic gene circuits for cell biology applications. Nat. Rev. Mol. Cell Biol. 19, 507-525 (2018).
41. Peterson, J. L. Petri nets. ACM Comput. Surv. 9, 223-252 (1977).
42. Chaouiya, C. Petri net modelling of biological networks. Brief. Bioinform. 8, 210-219 (2007).
43. Herajy, M., Liu, F. & Heiner, M. Design patterns for the construction of computational biological models. Brief Bioinform. 25, bbae318 (2024).
44. Rahimi, I., Gandomi, A. H., Nikoo, M. R. & Chen, F. A comparative study on evolutionary multi-objective algorithms for next release problem. Appl. Soft Comput. 144, 110472 (2023).
45. Gong, M., Jiao, L., Du, H. & Bo, L. Multiobjective immune algorithm with nondominated neighbor-based selection. Evol. Comput. 16, 225-255 (2008).
46. Tebo, A. G. & Gautier, A. A split fluorescent reporter with rapid and reversible complementation. Nat. Commun. 10, 2822 (2019).
47. Haellman, V., Saxena, P., Jiang, Y. & Fussenegger, M. Rational design and optimization of synthetic gene switches for controlling cell-fate decisions in pluripotent stem cells. Metab. Eng. 65, 99-110 (2021).
48. Benzinger, D., Ovinnikov, S. & Khammash, M. Synthetic gene networks recapitulate dynamic signal decoding and differential gene expression. Cell Syst 13, 353-364.e6 (2022).
49. Dolberg, T. B. et al. Computation-guided optimization of split protein systems. bioRxiv 863530 (2019) doi:10.1101/863530.
50. Pinkard, H. et al. Pycro-Manager: open-source software for customized and reproducible microscope control. Nat. Methods 18, 226-228 (2021).
51. Repina, N. A., Johnson, H. J., McClave, T., Kane, R. S. & Schaffer, D. V. Protocol to Fabricate Engineered Illumination Devices for Optogenetic Control of Cellular Signaling Dynamics. STAR Protoc 1, 100141 (2020).
52. Teague, B. Cytoflow: A Python Toolbox for Flow Cytometry. bioRxiv 2022.07.22.501078 (2022) doi:10.1101/2022.07.22.501078.
53. Virtanen, P. et al. SciPy 1.0: fundamental algorithms for scientific computing in Python. Nat. Methods 17, 261-272 (2020).
54. Newville, M., Stensitzki, T., Allen, D. B. & Ingargiola, A. LMFIT: Non-Linear Least-Square Minimization and Curve-Fitting for Python. (2014). doi:10.5281/zenodo.11813.
55. Halton, J. H. On the efficiency of certain quasi-random sequences of points in evaluating multi-dimensional integrals. Numer. Math. (Heidelb.) 2, 84-90 (1960).
56. van der Walt, S. et al. scikit-image: image processing in Python. PeerJ 2, e453 (2014).
57. Harris, C. R. et al. Array programming with NumPy. Nature 585, 357-362 (2020).
58. The pandas development team. Pandas-Dev/Pandas: Pandas. (Zenodo, 2024). doi:10.5281/ZENODO.13819579.
59. Hunter, J. D. Matplotlib: A 2D Graphics Environment. Comput. Sci. Eng. 9, 90-95 (2007).

What is claimed is:

1. A system for frequency-modulated (FM) control of cellular functions in a mammalian cell, comprising:
an optogenetic circuit comprising one or both of a first light-responsive protein interaction module and a second light-responsive protein interaction module, wherein:
the first light-responsive protein interaction module comprises a dimerization system responsive to high-frequency blue light stimulation (dense input), and
the second light-responsive protein interaction module comprises a combined dissociation and dimerization system with differential reversion kinetics responsive to low-frequency blue light stimulation (sparse input), wherein the optogenetic circuit is configured to decode a frequency of light input and generate distinct intracellular outputs corresponding to the frequency of light input, including at least one of:
(i) protein interaction domains operatively linked to the optogenetic circuit, the protein interaction domains enabling frequency-dependent control of protein-protein interactions;
(ii) a synthetic gene construct operatively linked to the optogenetic circuit, the synthetic gene construct comprising one or more regulatory elements that enable selective gene transcription in response to either high-frequency or low-frequency stimulation; or
(iii) a cell-surface signaling component operatively linked to the optogenetic circuit, the cell-surface signaling component enabling frequency-dependent control over cell-cell communication.

2. The system of claim 1, wherein the optogenetic circuit is a computationally optimized signaling motif configured to decode the frequency of light input and generate distinct intracellular outputs corresponding to the frequency of light input.

3. The system of claim 2, wherein the signaling motif comprises a computationally evolved network of ordinary differential equations optimized using a multi-objective evolutionary algorithm to balance simplicity and FM decoding fidelity.

4. The system of claim 1, wherein the first light-responsive protein interaction module comprises an iLID/sspB pair configured to dimerize under continuous or high-frequency blue light.

5. The system of claim 1, wherein the second light-responsive protein interaction module comprises a LOV/Zdk pair configured to dissociate under continuous or high-frequency blue light and re-associate under sparse or no light.

6. The system of claim 1, wherein said synthetic gene construct comprises a DNA-binding domain.

7. The system of claim 1, wherein the synthetic gene construct comprises a dual promoter system configured to independently drive expression of a first gene in response to dense input and a second gene in response to sparse input.

8. The system of claim 1, further comprising a fluorescent reporter construct, wherein frequency-modulated gene expression results in detectable expression of at least two different fluorescent proteins corresponding to dense and sparse light inputs.

9. The system of claim 1, wherein the synthetic gene construct encodes a tumor-associated antigen for presentation on a cell membrane, enabling frequency-dependent recognition and targeting by chimeric antigen receptor (CAR) T cells.

10. The system of claim 1, wherein the cell-surface signaling component enables paracrine signaling that induces differentiation in co-cultured receiver cells through frequency-dependent expression of synthetic ligands.

11. The system of claim 10, wherein the synthetic ligands are configured to engage engineered synNotch receptors in receiver cells to induce cell fate transitions.

12. The system of claim 1, wherein the optogenetic circuit is an FM decoder that exhibits differential output across a range of light input frequencies within a range of 0.05 Hz to 1 Hz.

13. The system of claim 12, wherein the FM decoder is further adapted to enable multiplexed control of gene expression in a single mammalian cell.

14. The system of claim 1, wherein the system is implemented in HEK293T cells, K562 tumor cells, or C3H fibroblast cells.

15. A method for frequency-modulated control of cellular functions in a mammalian cell, comprising:
(a) providing the mammalian cell with an optogenetic circuit comprising one or both of a first light-responsive protein interaction module comprising a dimerization system and a second light-responsive protein interaction module comprising a combined dissociation and dimerization system with differential reversion kinetics;
(b) exposing the mammalian cell to blue light at a first frequency to selectively activate the first light-responsive protein interaction module and generate a first intracellular output; and
(c) exposing the mammalian cell to blue light at a second frequency, different from the first frequency, to selectively activate the second light-responsive protein interaction module and generate a second intracellular output distinct from the first intracellular output.

16. The system of claim 1, wherein the second light-responsive protein interaction module comprises a fast-reverting dissociation component and a slow-reverting dimerization component, wherein a kinetic mismatch between reversion rates of the fast-reverting dissociation component and the slow-reverting dimerization component creates a temporal window during pulsed illumination in which both components achieve a signaling-active configuration.

17. The system of claim 16, wherein the fast-reverting dissociation component comprises a LOVfast/Zdk pair and the slow-reverting dimerization component comprises an iLIDslow/sspB pair.

18. The system of claim 6, wherein the DNA-binding domain is operatively linked to a light-inducible transcriptional activator, such that frequency-dependent dimerization or dissociation of the optogenetic circuit recruits the transcriptional activator to the DNA-binding domain to drive expression of a gene of interest.

19. The method of claim 15, wherein the first frequency is approximately 1 Hz and the second frequency is approximately 0.25 Hz.

20. The method of claim 15, wherein the first intracellular output or the second intracellular output comprises frequency-dependent expression of a tumor-associated antigen on a surface of the mammalian cell, and the method further comprises contacting the mammalian cell with chimeric antigen receptor (CAR) T cells that selectively target the tumor-associated antigen.
